(19) United States
(12) Patent Application Publication
(10) Pub. No.: US 2011/0048980 A1
(43) Pub. Date: Mar. 3, 2011

(54) Fatty Acid Oxidation Inhibitors Treating Hyperglycemia and Related Disorders

(75) Inventor: Leo J. Seman, Cheshire, CT (US)

(73) Assignee: Symcopeia Company, Carmel, IN (US)

(21) Appl. No.: 12/530,252

(22) PCT Filed: Sep. 13, 2007
(86) PCT No.: PCT/US2007/019852

§ 371 Date: May 21, 2010

Related U.S. Application Data
(60) Provisional application No. 60/905,934, filed on Mar. 9, 2007.

(51) Int. Cl. B65D 69/00; A61K 31/495; A61K 31/155; A61P 3/10

(52) U.S. Cl. 206/223; 514/252.12; 514/635

(57) ABSTRACT

The invention relates to a methods, compositions and kits for treating hyperglycemia and related disorders, such as type 2 diabetes mellitus, impaired glucose tolerance, diabetic retinopathy, diabetic nephropathy and diabetic neuropathy, and to methods, compositions and kits for treating erectile dysfunction. The methods comprise administering an inhibitor of fatty acid oxidation to a subject in need thereof. In some embodiments, trimetazidine and metformin or a phosphodiesterase 5 inhibitor are administered.

### RELATED APPLICATIONS

This application claims the benefit of U.S. Provisional Application No. 60/905,934 filed Mar. 9, 2007, the entire teachings of which are incorporated herein by reference.

### BACKGROUND OF THE INVENTION

Type 2 diabetes (T2DM) is the most common form of diabetes and it occurs when the body does not produce sufficient amounts of insulin, a hormone that cause cells to take up glucose from the blood, or when cells are resistant to the biological effects of insulin. As a result, cells are starved of glucose, their basic energy source, and the levels of glucose in the blood build up to pathological levels. T2DM has reached pandemic levels, affecting over 150 million people worldwide. The prevalence of the disease is expected to increase to 300 million people by the year 2025. Although there is the potential to reduce this pandemic by weight control, the increased availability of low-cost/high caloric food and sedentary lifestyles have fueled obesity and subsequent T2DM.

T2DM can lead to serious life threatening sequelae, which include T2DM-associated diseases, such as cardiovascular disease (leading to an increased prevalence of myocardial infarction, sudden death, acute coronary syndromes, stroke and chronic renal failure), retinopathy, nephropathy and neuropathy.

Patients with T2DM have an increased risk of cardiovascular disease that has been estimated to be 2-4 fold greater than subjects without T2DM. Although it is not clear how T2DM increases this risk, it is clear that this population often has a constellation of hypertriglyceridemia, low HDL, hypertension and impaired glucose tolerance for several years prior to developing T2DM. This constellation, known as the Metabolic Syndrome can also be defined as a collection of cardiovascular risk factors which contribute to the overall increased risk of coronary heart disease (CHD) in the T2DM population.

Additionally, glycation, the process of non-enzymatic glucosylation of proteins that occurs when glucose levels are high, can lead to abnormal function of structural proteins and other proteins that normally have a long half life, and contribute to the pathogenesis of T2DM-associated diseases, such as cardiovascular disease. For example, glycation can lead to tissue hypoxia, which can induce pathologic changes which are presumed to account for many of the T2DM-associated diseases such as CHD, retinopathy, nephropathy, and neuropathy. Down-stream effects of hypoxia include stimulation of phosphorylation of vascular growth factors which can contribute to neovascularization, endothelial dysfunction, atherosclerosis and ischemia. Endothelial dysfunction in conjunction with atherosclerosis results in decreased perfusion of tissues and creates further levels of tissue ischemia and hypoxia. This is particularly problematic for the heart since cardiac muscle derives most of its energy from oxidation of free fatty acids and has one of the highest consumptions of oxygen in the body. Cardiac hypoxia is further complicated in T2DM patients because there is very limited circulation and perfusion of cardiac tissue.

Present therapies to treat T2DM reduce hyperglycemia, but do not fully control glucose or lipid metabolism or the life threatening T2DM-associated diseases. Current therapies for T2DM urge weight loss, a low glycemic index diet, and exercise, and may include the administration of glucose reducing agents, such as sulfonylureas, biguanides (metformin), PPAR agonists (such as thiazolidinediones), GLP-1 analogs (Byetta, exendin), dipeptidyl peptiase inhibitors (e.g., sitagliptin phosphate, vildagliptin), insulins or insulin analogs, lipid reducing agents (e.g., a statin), and/or antihypertensive agents (e.g., angiotensin converting enzyme (ACE) inhibitor, (AT2) Angiotensin 2 receptor inhibitor). This therapeutic constellation is designed to reduce glucose levels and to reduce the risk factors (e.g., hypertriglyceridemia, low HDL, hypertension and impaired glucose tolerance) associated with, and which contribute to, the morbidity and mortality of T2DM.

The use of cardiovascular risk-reducing therapies as adjunctive therapy secondary to diabetes therapy can reduce morbidity and mortality in T2DM patients due to cardiovascular disease. However, glucose-lowering therapies have been less successful in demonstrating a reduction in either cardiovascular or overall morbidity and mortality in T2DM patients. For example, the United Kingdom Prospective Diabetes Study (UKPDS) demonstrated that glucose control with metformin reduced cardiovascular events while glucose control with sulfonylureas and insulin did not (Diabetes Metab., 29(4 Pt 2):6S36-43 (2003)). The PROspective PioglitAzone Clinical Trial In MacroVascular Events (PROACTIVE) study demonstrated that adding pioglitazone to other glucose lowering drugs to achieve equivalent glucose-lowering, reduced the risk of several coronary heart disease (CHD) events, but increased the risk of congestive heart failure (CHF) 2-3 fold. The Diabetes ReEduction Approaches with ramipril and rosiglitazone Medications (DREAM) study demonstrated that treating impaired glucose tolerance (IGT) patients with rosiglitazone reduced the onset of T2DM by 60% as compared to placebo, but did not statistically reduce CHD events, which trended to a relative risk of 1.37 (RR=1.37) and increased the incidence of CHF by 7-fold in this pre-diabetic population.

In view of the serious life threatening sequelae of T2DM, which are not improved by therapy with many glucose lowering agents, such as sulfonylureas and insulin, a need exists for therapy that effectively lowers glucose levels (as predicted by the reduction in the percentage of glycated hemoglobin A1c (% HbA1c)) and reduces the CHD risk in diabetic patients. New approaches to lower glucose and simultaneously reduces coronary heart disease are needed.

Persistent hyperglycemia (e.g., chronic hyperglycemia), as measured by an elevated % HbA1c, is predictive of increased diabetic retinopathy. Diabetic proliferative retinopathy is characterized by the abnormal growth of new blood vessels on the retina, which is speculated to be initiated by hypoxia to the retina. The hypoxia is speculated to occur because of changes in the connective tissues of the basement membranes of the retina vasculature. Diabetic retinopathy presents clinically with neovascularization and evidence of retinal bleeds. Other components of diabetic retinopathy include non-proliferative diabetic retinopathy and macular edema. These are also related to the microvascular damage and hypoxia associated with diabetes. The Diabetes Control and Complications Trial (DCCT) clearly demonstrated that % HbA1c positively correlated with the incidence and severity of diabetic retinopathy. Despite efforts to reduce the incidence of diabetic retinopathy by setting more aggressive goals in lowering % HbA1c, diabetic retinopathy remains a major cause of blindness.

One approach for treating diabetic retinopathy that is in development concerns reducing vascular endothelial growth factor (VEGF), a stimulant of neovascularization. However, with this approach, there remain concerns that the hypoxic retina will degenerate without neovascularization. New therapies are needed to treat and/or prevent diabetic retinopathy.

Normal renal physiology is dependent on the integrity of the filtering function of the glomerulus, which serves as the renal filter. The presence of persistent hyperglycemia (e.g., chronic hyperglycemia) associated with T2DM or not associated with T2DM, increases the gradient of flow of fluids across the glomerulus resulting in hyperfiltration. This hyperfiltration, in combination with vascular inflammation and hypoxia, results in injury and disruption of the integrity of the glomerular membrane. Injury and disruption of the glomerular membrane results in impaired renal function, which can be diagnosed by the presence of increased protein excretion in the urine (e.g., in the forms of microscopic levels of albumin (microalbuminuria) or overt levels of albumin (proteinuria)), or by impaired glomerular filtration as measured by creatinine clearance or glomerular filtration rate.

Treatment for diabetic nephropathy consists of controlling blood sugars and controlling blood pressure (BP) with drugs that block the effect of angiotensin 2 (a neurohormone that controls vasoconstriction, water and salt absorption). Aggressive blood sugar control and BP control slow nephropathy progression, but disease progression persists. Vascular charge reducing agents such as sulodexide are being tested for treatment of diabetic nephropathy. New therapies are needed to treat and/or prevent diabetic nephropathy.

Diabetic peripheral neuropathy is a microvascular complication that occurs in almost half of the patients with T2DM (Dyck, P J., et al., Neurology 43:817-824 (1993)) in the US and Europe. (Tesfaye, S., et al., Diabetologia 39:1377-1384 (1996)). Diabetic peripheral neuropathy is a significant risk factor for foot ulcerations and lower extremity amputations. (Manes, C., et al., Wounds 14:11-15 (2002)). Improvement of sensory symptoms and quality of life is recognized as an important clinical endpoint, particularly if associated with improved nerve function. (Apfel, S. C., J. Neurol. Sci. 189:3-5 (2001)). Strict normalization of hyperglycemia and reduction of % HbA1c is an effective preventive measure in only about 50% of patients with diabetes. Current treatments for diabetic peripheral neuropathy focus on masking pain, but do not address the underlying pathophysiology, which is an impairment of microvascular function of the blood vessels of the peripheral nerves (Bolton, A. J., Diabetes Care 28:956-962 (2005)) resulting in hypoxic damage to the nerve. New therapies for diabetic peripheral neuropathy are needed.

Trimetazidine is a compound that has been used as an antianginal agent. Trimetazidine is thought to be a 3-KAT (3-Ketoacyl coA Thiolase) inhibitor, and has been extensively studied in cardiac cell cultures and ex vivo heart perfusion models. It has been hypothesized that cardiac-type muscle cells utilize less oxygen when exposed to trimetazidine because trimetazidine inhibits the oxidation of long-chain fatty acids, which is the primary metabolic source of energy in cardiac muscle cells. Trimetazidine is hypothesized to increases glucose uptake and oxidation in cardiac muscle cells, as an assumed compensatory mechanism to supply energy to the cells. It has also been hypothesized that glucose oxidation in cardiac muscle cells can provide a similar amount of ATP energy units while utilizing less oxygen than long-chain fatty acyl oxidation. Additionally, it has been theorized that long-chain fatty acyl oxidation can be cardiotoxic, leading to systolic dysfunction and less efficient utilization of energy (and less efficient oxygen consumption). Thus, it is believed that trimetazidine can reduce oxygen requirements in cardiac muscle and therefore, can provide cardiovascular protective effects under hypoxic and/or ischemic conditions, such as in angina pectoris. Trimetazidine has been suggested as adjunctive therapy secondary to diabetes therapy in patients with T2DM and ischemic cardiomyopathy. (See, Fragasso, et al., Am. Heart J., 146:e18 (2003); Monti, et al., Am. J. Physiol, Endocrinol. Metab. 209:54-59, (2006); and Fragasso, et al., Heart Metab, 30:21-24 (2006)). The use of trimetazidine under these circumstances has been shown to improve cardiac function. Trimetazidine has also been shown to protect against ischemic injuries in experimental models of ischemic retina and renal injury/ischemia. (See, Payet et al., J. Ocular. Pharmacol, Therapeutics, 20(1): 85-92 (2004); and Domanski, et al., Eur. J. Pharm. Sci., 27(4):320-7 (March 2006; Epub January 2006)).

Erectile impotence or erectile dysfunction (ED) is the inability to obtain or sustain an erection adequate for intercourse. Its prevalence is claimed to be between 2 and 7% of the human male population, increasing with age, up to 50 years, and between 18 and 75% between 55 and 80 years of age. In the USA alone, for example, it has been estimated that there are up to 10 million impotent males, with the majority suffering from problems of physiologic rather than of psychogenic origin. The seriousness of this problem is demonstrated by the commercial success of agents for treating ED.

Erection is caused by the vasodilatory effects of cGMP, the production of which is stimulated by the release of nitric oxide (NO). NO release in the corpus cavernosum is induced by neuronal impulses during sexual stimulation. Current ED therapeutics, such as tadalafil, sildenafil and vardenafil, do not alter the amount of cGMP produced, but inhibit the phosphodiesterase 5 enzyme which degrades cGMP, thereby increasing levels of cGMP. However, these types of therapeutics are not suitable for all patients as phosphodiesterase 5 inhibitors are contraindicated for patients using organic nitrates, and are not effective if the patient has impaired production of NO in the corpus cavernosum. New approaches to treat ED are needed

### SUMMARY OF THE INVENTION

The invention relates to a method for treating hyperglycemia (e.g., chronic hyperglycemia, hyperglycemia with diabetes, hyperglycemia without diabetes), type 2 diabetes mellitus, impaired glucose tolerance and/or metabolic syndrome comprising administering to a subject in need thereof a therapeutically effective amount of an inhibitor of fatty acid oxidation. In some embodiments, the method is a method of front line therapy for treating hyperglycemia, type 2 diabetes mellitus, impaired glucose tolerance and/or metabolic syndrome. In some embodiments, the fatty acid oxidation inhibitor is not ranolazine or etomoxir. The inhibitor of fatty acid oxidation can be a 3-KAT inhibitor, such as trimetazidine.

The invention relates to the use of an inhibitor of fatty acid oxidation for the manufacture of a medicament for the treatment of hyperglycemia (e.g., chronic hyperglycemia, hyperglycemia with diabetes, hyperglycemia without diabetes), type 2 diabetes mellitus, impaired glucose tolerance and/or metabolic syndrome. The inhibitor of fatty acid oxidation can be a 3-KAT inhibitor, such as trimetazidine.

The invention relates to a pharmaceutical composition for the treatment of hyperglycemia (e.g., chronic hyperglycemia, hyperglycemia with diabetes, hyperglycemia without diabetes), type 2 diabetes mellitus, impaired glucose tolerance and/or metabolic syndrome, comprising as an active ingredient an inhibitor of fatty acid oxidation. The inhibitor of fatty acid oxidation can be a 3-KAT inhibitor, such as trimetazidine.

The invention relates to a method for treating hyperglycemia (e.g., chronic hyperglycemia, hyperglycemia with diabetes, hyperglycemia without diabetes), type 2 diabetes mellitus, impaired glucose tolerance and/or metabolic syndrome comprising administering to a subject in need thereof a therapeutically effective amount of an inhibitor of fatty acid oxidation and an inhibitor of hepatic glucose output. The inhibitor of fatty acid oxidation can be a 3-KAT inhibitor, such as trimetazidine. The inhibitor of hepatic glucose output can be metformin. In one embodiment, trimetazidine is administered at about 30 to about 180 mg/day and metformin is administered at about 1000-2550 mg/day.

The invention relates to use of an inhibitor of fatty acid oxidation and an inhibitor of hepatic glucose output for the manufacture of a medicament for the treatment of hyperglycemia (e.g., chronic hyperglycemia, hyperglycemia with diabetes, hyperglycemia without diabetes), type 2 diabetes mellitus, impaired glucose tolerance and/or metabolic syndrome. The inhibitor of fatty acid oxidation can be a 3-KAT inhibitor, such as trimetazidine. The inhibitor of hepatic glucose output can be metformin. In one embodiment, the medicament comprises a daily dose of about 30 to about 180 mg of trimetazidine and about 1000-2550 mg of metformin.

The invention relates to a pharmaceutical composition for treating hyperglycemia (e.g., chronic hyperglycemia, hyperglycemia with diabetes, hyperglycemia without diabetes), type 2 diabetes mellitus, impaired glucose tolerance and/or metabolic syndrome comprising as active ingredients an inhibitor of fatty acid oxidation and an inhibitor of hepatic glucose output. The inhibitor of fatty acid oxidation can be a 3-KAT inhibitor, such as trimetazidine. The inhibitor of hepatic glucose output can be metformin. In one embodiment, the pharmaceutical composition is for the administration of about 30 to about 180 mg/day trimetazidine and about 1000-2550 mg/day metformin.

The invention relates to a method for treating hyperglycemia (e.g., chronic hyperglycemia, hyperglycemia with diabetes, hyperglycemia without diabetes), type 2 diabetes mellitus, impaired glucose tolerance and/or metabolic syndrome comprising administering to a subject in need thereof a therapeutically effective amount of an inhibitor of fatty acid oxidation and another antidiabetic agent. The inhibitor of fatty acid oxidation can be a 3-KAT inhibitor, such as trimetazidine. The antidiabetic agent can be a dipeptidyl peptidase inhibitor (e.g., a dipeptidyl peptidase IV inhibitor, such as sitagliptin or vildagliptin). In some embodiments, the inhibitor of fatty acid oxidation is trimetazidine, and the other antidiabetic agent is selected from the group consisting of sitagliptin, vildagliptin, acarbose, pioglitazone, and rosiglitazone.

The invention relates to use of an inhibitor of fatty acid oxidation and another antidiabetic agent for the manufacture of a medicament for the treatment of hyperglycemia (e.g., chronic hyperglycemia, hyperglycemia with diabetes, hyperglycemia without diabetes), type 2 diabetes mellitus, impaired glucose tolerance and/or metabolic syndrome. The inhibitor of fatty acid oxidation can be a 3-KAT inhibitor, such as trimetazidine. The antidiabetic agent can be a dipeptidyl peptidase inhibitor, such as sitagliptin or vildagliptin. In some embodiments, the inhibitor of fatty acid oxidation is trimetazidine, and the other antidiabetic agent is selected from the group consisting of sitagliptin, vildagliptin, acarbose, pioglitazone, and rosiglitazone.

The invention relates to a pharmaceutical composition for treating hyperglycemia (e.g., chronic hyperglycemia, hyperglycemia with diabetes, hyperglycemia without diabetes), type 2 diabetes mellitus, impaired glucose tolerance and/or metabolic syndrome comprising as active ingredients an inhibitor of fatty acid oxidation and another antidiabetic agent. The inhibitor of fatty acid oxidation can be a 3-KAT inhibitor, such as trimetazidine. The antidiabetic agent can be a dipeptidyl peptidase inhibitor, such as sitagliptin or vildagliptin. In some embodiments, the inhibitor of fatty acid oxidation is trimetazidine, and the other antidiabetic agent is selected from the group consisting of sitagliptin, vildagliptin, acarbose, pioglitazone, and rosiglitazone.

The invention relates to a method for treating diabetic retinopathy, diabetic nephropathy and/or diabetic neuropathy comprising administering to a subject in need thereof a therapeutically effective amount of an inhibitor of fatty acid oxidation. In particular embodiments, the inhibitor of fatty acid oxidation is a 3-KAT inhibitor (e.g., trimetazidine, ranolazine).

The invention relates to use of an inhibitor of fatty acid oxidation for the manufacture of a medicament for the treatment of diabetic retinopathy, diabetic nephropathy and/or diabetic neuropathy. In particular embodiments, the inhibitor of fatty acid oxidation is a 3-KAT inhibitor (e.g., trimetazidine, ranolazine).

The invention relates to a pharmaceutical composition for treating diabetic retinopathy, diabetic nephropathy and/or diabetic neuropathy comprising as an active ingredient an inhibitor of fatty acid oxidation. In particular embodiments, the inhibitor of fatty acid oxidation is a 3-KAT inhibitor (e.g., trimetazidine, ranolazine).

The invention relates to a method for treating diabetic retinopathy, diabetic nephropathy and/or diabetic neuropathy comprising administering to a subject in need thereof a therapeutically effective amount of an inhibitor of fatty acid oxidation and an inhibitor of hepatic glucose output. The inhibitor of fatty acid oxidation can be a 3-KAT inhibitor, such as trimetazidine. The inhibitor of hepatic glucose output can be metformin. In one embodiment, trimetazidine is administered at about 30 to about 180 mg/day and metformin is administered at about 1000-2550 mg/day.

The invention relates to use of an inhibitor of fatty acid oxidation and an inhibitor of hepatic glucose output for the manufacture of a medicament for the treatment of diabetic retinopathy, diabetic nephropathy and/or diabetic neuropathy. The inhibitor of fatty acid oxidation can be a 3-KAT inhibitor, such as trimetazidine. The inhibitor of hepatic glucose output can be metformin. In one embodiment, trimetazidine is administered at about 30 to about 180 mg/day and metformin is administered at about 1000-2550 mg/day.

The invention relates to a pharmaceutical composition for treating diabetic retinopathy, diabetic nephropathy and/or diabetic neuropathy comprising as active ingredients an inhibitor of fatty acid oxidation and an inhibitor of hepatic glucose output. The inhibitor of fatty acid oxidation can be a 3-KAT inhibitor, such as trimetazidine. The inhibitor of hepatic glucose output can be metformin. In one embodiment, trimetazidine is administered at about 30 to about 180 mg/day and metformin is administered at about 1000-2550 mg/day.

The invention relates to a kit for treating hyperglycemia (e.g., chronic hyperglycemia, hyperglycemia with diabetes, hyperglycemia without diabetes), impaired glucose tolerance, T2DM, and/or sequelae of hyperglycemia and/or T2DM including cardiovascular disease (e.g., leading to an increased prevalence of myocardial infarction, sudden death, acute coronary syndromes, stroke and chronic renal failure), diabetic retinopathy, diabetic nephropathy and diabetic neuropathy. The kit can contain a first pharmaceutical composition comprising an inhibitor of fatty acid oxidation, and a second pharmaceutical composition comprising an inhibitor of hepatic glucose output or another antidiabetic agent.

The invention relates to a method for treating erectile dysfunction comprising administering to a subject in need thereof a therapeutically effective amount of an inhibitor of fatty acid oxidation. The inhibitor of fatty acid oxidation can be a 3-KAT inhibitor, such as trimetazidine.

The invention relates to use of an inhibitor of fatty acid oxidation for the manufacture of a medicament for the treatment of erectile dysfunction. The inhibitor of fatty acid oxidation can be a 3-KAT inhibitor, such as trimetazidine.

The invention relates to a pharmaceutical composition for treating erectile dysfunction comprising as an active ingredient an inhibitor of fatty acid oxidation. The inhibitor of fatty acid oxidation can be a 3-KAT inhibitor, such as trimetazidine.

The invention relates to a method for treating erectile dysfunction comprising administering to a subject in need thereof a therapeutically effective amount of an inhibitor of fatty acid oxidation and an inhibitor of cGMP degradation. The inhibitor of fatty acid oxidation can be a 3-KAT inhibitor, such as trimetazidine. The inhibitor of cGMP degradation can be a phosphodiesterase inhibitor (e.g., an inhibitor of phosphodiesterase 1, 2, 3, 4, 5, 6, 7, 8, 9, 10 or 11). Preferably, the phosphodiesterase inhibitor is a phosphodiesterase 5 inhibitor such as tadalafil, sildenafil or vardenafil.

The invention relates to use of an inhibitor of fatty acid oxidation and an inhibitor of cGMP degradation for the manufacture of a medicament for the treatment of erectile dysfunction. The inhibitor of fatty acid oxidation can be a 3-KAT inhibitor, such as trimetazidine. The inhibitor of cGMP degradation can be a phosphodiesterase inhibitor (e.g., an inhibitor of phosphodiesterase 1, 2, 3, 4, 5, 6, 7, 8, 9, 10 or 11). Preferably, the phosphodiesterase inhibitor is a phosphodiesterase 5 inhibitor such as tadalafil, sildenafil or vardenafil.

The invention relates to a pharmaceutical composition for treating erectile dysfunction comprising as an active ingredient an inhibitor of fatty acid oxidation and an inhibitor of cGMP degradation. The inhibitor of fatty acid oxidation can be a 3-KAT inhibitor, such as trimetazidine. The inhibitor of cGMP degradation can be a phosphodiesterase inhibitor (e.g., an inhibitor of phosphodiesterase 1, 2, 3, 4, 5, 6, 7, 8, 9, 10 or 11). Preferably, the phosphodiesterase inhibitor is a phosphodiesterase 5 inhibitor such as tadalafil, sildenafil or vardenafil.

The invention also relates to a kit for treating ED. The kit can comprise a first pharmaceutical composition comprising an inhibitor of fatty acid oxidation, and a second pharmaceutical composition comprising an agent that inhibits cGMP degradation (e.g., a phosphodiesterase inhibitor, such as a phosphodiesterase 5 inhibitor).

The invention relates to a pharmaceutical composition comprising trimetazidine and metformin and a physiologically acceptable carrier. The invention relates to a pharmaceutical composition comprising ranolazine and metformin and a physiologically acceptable carrier. The invention relates to a pharmaceutical composition comprising etomoxir and metformin and a physiologically acceptable carrier.

The invention also relates to a method for treating a patient in need thereof for metabolic syndrome or diabetes and endothelial dysfunction comprising administering a combination of two or more compounds selected from the group consisting of an HMG CoA reductase inhibitor, a partial fatty acid oxidation (“pFox”) inhibitor, one or more oral hypoglycemics, a protein kinase C inhibitor, and an acetyl-CoA carboxylase inhibitor. This aspect of the invention is also the subject of disclosure and claims in U.S. patent application Ser. No. 11/373,658 (US 2006/0205727 A1). The entire teachings of U.S. patent application Ser. No. 11/373,658 (US 2006/0205727 A1) are incorporated herein by reference.

### DETAILED DESCRIPTION OF THE INVENTION

Current front line therapies for treating hyperglycemia and T2DM focus on reducing glucose levels (e.g., as predicted by reduction in % HbA1c). Because patients with hyperglycemia and T2DM are at increased risk for morbidity and mortality due to life threatening sequelae, such as T2DM-associated cardiovascular disease, retinopathy, nephropathy and neuropathy, adjunctive therapy that seeks to reduces risk factors for such diseases (e.g., lipid reducing agents, antihypertensive agents, antianginal agents) may be administered secondary to glucose lowering therapy. The addition of adjunctive therapy can reduce morbidity and mortality in hyperglycemic or T2DM patients due to, for example, cardiovascular disease. However, there are currently no front line therapies that effectively lower glucose levels and also reduce the incidence of life threatening sequelae of hyperglycemia and T2DM, or that reduce morbidity and mortality due to sequelae of hyperglycemia and T2DM, including T2DM-associated cardiovascular disease, retinopathy, nephropathy and neuropathy, independent of its effect on hyperglycemia.

The invention relates to methods, compositions and kits for treating hyperglycemia (e.g., chronic hyperglycemia, hyperglycemia with diabetes, hyperglycemia without diabetes), impaired glucose tolerance, T2DM, metabolic syndrome, and/or sequelae of hyperglycemia and/or T2DM, including cardiovascular disease (e.g., leading to an increased prevalence of myocardial infarction, sudden death, acute coronary syndromes, stroke and chronic renal failure), diabetic retinopathy, diabetic nephropathy and diabetic neuropathy. The invention provides improved therapy, including front line therapy, that effectively lowers glucose levels. The improved therapy described herein can also reduce the incidence of life threatening sequelae of hyperglycemia and T2DM, and/or reduce morbidity and mortality due to sequelae of hyperglycemia and T2DM, including T2DM-associated cardiovascular disease, retinopathy, nephropathy and neuropathy. The invention also relates to methods, compositions and kits for treating ED.

3-Ketoacetyl A-CoA thiolase (3-KAT) is a key enzyme in fatty acid beta oxidation. Inhibition of 3-KAT with trimetazidine has been reported to inhibit fatty acid oxidation and increase glucose uptake and oxidation in cardiac muscle cells, thereby reducing cardiac oxygen demands, and improving cardiac function in patients with T2DM and ischemic cardiomyopathy. (See, Fragasso, et al., Am. Heart J., 146:e18 (2003); Monti, et al., Am. J. Physiol, Endocrinol. Metab. 209:54-59, (2006); and Fragasso, et al., Heart Metab, 30:21-24 (2006)). In one study of adjunctive trimetazidine therapy, trimetazidine was administered to male diabetic patients with ischemic heart disease who were also receiving ACE inhibitors and β-blockers to manage their heart disease. (Monti, et al., Am. J. Physiol, Endocrinol. Metab. 209:54-59, (2006)). In this study, a 12% reduction in fasting blood glucose after 15 days of treatment with trimetazidine (20 mg by mouth, three times daily) was observed. (Id.). This observation is consistent with the observation of Fragasso, who reported a reduction in glycohemoglobin A1c of 0.8% when compared to placebo when trimetazidine (20 mg by mouth, three times daily) was added to preexisting cardiovascular therapy in T2DM patients with hypokinetic cardiomyopathy. (Fragasso, et al., Am. Heart J. 146:e18 (2003)). Additionally, an increase in glucose oxidation correlated positively with an increase in the generation of the vasodilator cGMP (cyclic guanosine monophosphate). (Monti, et al., Am. J. Physiol, Endocrinol. Metab. 209:54-59, (2006)). The vasodilatory properties of cGMP are beneficial in improving coronary artery blood flow, as well as improving erectile dysfunction particularly in the setting of a phosphodiesterase inhibitor (e.g., a phosphodiesterase 5 inhibitor such as vardenafil, sildenafil, or tadalafil), which prevents the degradation of cGMP to GMP (guanosine monophosphate). It remains unclear whether the improvement in plasma glucose levels observed in these studies relates directly to improved cardiac muscle and skeletal muscle uptake and oxidation of glucose, or to the improvement in cardiac output which occurs with trimetazidine treatment. Improved cardiac output results in improved organ perfusion and subsequently increased uptake of oxygen and glucose by end organs, and also results in improved renal clearance, which could account for the observed reductions in glucose levels.

Accordingly, it has been suggested that trimetazidine may be suitable for adjunctive therapy for T2DM patients with ischemic cardiomyopathy. However, trimetazidine has not been suggested for front line therapy for hyperglycemia, T2DM, T2DM-associated diseases, metabolic syndrome, or for treating ED. (See, Fragasso, et al., Am. Heart J., 146:e18 (2003); Monti, et al., Am. J. Physiol, Endocrinol. Metab. 209:54-59, (2006); and Fragasso, et al., Heart Metab, 30:21-24 (2006)).

As described herein, it has now been determined that inhibitors of fatty acid oxidation (e.g., trimetazidine) can produce a variety of therapeutic effects that have benefits for treating hyperglycemia, T2DM, impaired glucose tolerance, metabolic syndrome and related disorders. In particular, inhibitors of fatty acid oxidation (e.g., trimetazidine) can be administered as front line therapy to lower glucose levels and to also target other metabolic disorders that perpetuate and contribute to the pathology of hyperglycemia, T2DM, impaired glucose tolerance, metabolic syndrome and related disorders. As a result, inhibitors of fatty acid oxidation (e.g., trimetazidine) can be administered in accordance with the methods described herein to reduce glucose levels and to concurrently inhibit the development and progression of life-threatening sequelae of hyperglycemia and/or T2DM including cardiovascular disease (e.g., leading to an increased prevalence of myocardial infarction, sudden death, acute coronary syndromes, stroke and chronic renal failure), diabetic retinopathy, diabetic nephropathy, diabetic neuropathy, and ED.

Administration of inhibitors of fatty acid oxidation (e.g., trimetazidine) in accordance with the invention reduces circulating glucose (e.g., as determined by % HbA1c). This reduction in glucose is believed to be the result of increased uptake of glucose by skeletal muscle, which is the major organ system for glucose uptake and for maintaining glucose homeostasis. As a result of the reduction in circulating glucose, there can be a reduction in the glycation of proteins, particularly structural proteins and proteins with long half-lives. Proteins that are glycated when glucose levels rise often compose the fibrous and connective tissues that make up the basement membranes of blood vessels, e.g., microvascular and macrovascular vessels. The glycation of such proteins can adversely impact the exchange of oxygen and nutrients through the vessels to the underlying tissues and organs. By reducing glucose levels and subsequent glycation of proteins, inhibitors of fatty acid oxidation (e.g., trimetazidine) can improve vascular function, and perfusion and oxygenation of end organs.

This improvement in glucose and oxygen handling at the cellular and tissue level can result in a decrease in oxygen free radicals and resultant oxidative stress. Loss of oxidative stress at the tissue level decreases the generation of endothelial tissue derived factors such as endothelin-1 (ET-1), mitogen-activated protein (MAP) kinase, and tissue derived growth factors. In addition, a reduction of the levels of oxygen free radicals at the tissue interface can increase the genetic expression of endothelial nitric oxide synthase (eNOS), and thereby increase nitric oxide, a chemical substance that enhances vasodilation. This reduction in endothelial derived factors can reduce the recruitment of inflammatory cells and hence reduces 1) local inflammation; 2) further oxidation of lipids; and 3) activation of the local coagulation pathways and activation of platelets.

These drug-induced changes improve vascular function and can also result in a decrease in angiogenesis, stabilization of atherosclerotic plaque, and an increase in vasodilation, with a subsequent increase in blood flow to end organs. It is well-recognized that atherosclerotic plaques contain connective tissues and fibrous tissues which can be glycated and subsequently signal inflammation and repair in a delicately stabilized system of atheroma, fibrin, platelets, macrophages, and immunocytes. It is also well-recognized that elements that destabilize this delicate milieu can stimulate plaque rupture, with the release of atheroma downstream from the original atherosclerotic plaques, that will stimulate platelet aggregation and the formation of a vessel-occluding athero-thrombus. Thus, inhibitor of fatty acid oxidation (e.g., trimetazidine) mediated improvement of vascular function and/or stabilization of atherosclerotic plaque is a significant benefit of the invention, and can reduce morbidity and mortality associated with hyperglycemia, T2DM, impaired glucose tolerance, metabolic syndrome and related disorders.

Administration of inhibitors of fatty acid oxidation (e.g., trimetazidine) in accordance with the invention reduces beta oxidation of free fatty acids, with a subsequent reduction in by-products of beta oxidation. Thus, the invention provides a method for reducing glucose and for addressing other metabolic abnormalities which contribute to hyperglycemia, T2DM, impaired glucose tolerance and related disorders.

By reducing beta oxidation of free fatty acids and reducing the amount of by-products of beta oxidation, which can promote inflammation and cell death, inflammation and cell damage or cell death can be reduced. There can also be a reduction in uptake of free fatty acids into non-adipocyte tissues and a reduction in intracellular triglyceride accumulation. This can result in normalized regulation of protein kinase C-zeta activity in non-adipocyte tissue, with a subsequent improvement in insulin sensitivity and signaling. In the liver, the improved insulin signaling can lead to a reduction in gluconeogenesis and glycogenolysis, and increased glycogen synthesis and conversion of glucose to glycogen. This can further decrease circulating glucose as determined by the percent glycated HbA1c.

The sum of effects that are induced by administration of an inhibitor of fatty acid oxidation (e.g., trimetazidine), include a reduction in the generation of oxidized fatty acid by-products, a reduction in plasma concentrations of glucose, a reduction in glycation of structural proteins in the microvasculature, and improvement in vascular function provide advantages not currently available from other glucose lowering agents. Administration of a fatty acid oxidation inhibitor also provides a superior activity profile for front line therapy for hyperglycemia, T2DM, impaired glucose tolerance, metabolic syndrome and related disorders. For example, in typical diabetic patients who are not as seriously cardiac compromised, such as patients in the studies by Monti and Fragasso (Monti, et al., Am. J. Physiol, Endocrinol. Metab. 209:54-59 (2006); and Fragasso, et al., Am. Heart J., 146:e18 (2003)), the sum of effects of trimetazidine alone, possibly when administered at higher more inhibitory doses than previously used (e.g., about 61 mg/day to about 200 mg/day), or in combination with a complimentary antidiabetic drug such as metformin, would: 1) inhibit the oxidation of long chain fatty acids in skeletal muscle; 2) increase the expression and translocation of glut4 glucose transfer; 3) increase the expression of uncoupling protein 3 which would shuttle free fatty acids out of the skeletal cell membrane; 4) produce a clinically relevant increase in uptake and utilization of glucose by the skeletal muscle; and 5) produce subsequent reductions in plasma glucose levels, leading to reductions in % HbA1c.

The invention relates to the use of an inhibitor of fatty acid oxidation, such as a 3-ketoacetyl A-CoA thiolase (3-KAT) inhibitor (e.g., trimetazidine, ranolazine), or carnitine palmitoyltransferase-1 (CPT-1) inhibitor (e.g., etomoxir), for the treatment of hyperglycemia (e.g., chronic hyperglycemia, hyperglycemia with diabetes, hyperglycemia without diabetes), impaired glucose tolerance, metabolic syndrome, T2DM, sequelae of hyperglycemia and/or T2DM including cardiovascular disease (e.g., leading to an increased prevalence of myocardial infarction, sudden death, acute coronary syndromes, stroke and chronic renal failure), diabetic retinopathy, diabetic nephropathy and diabetic neuropathy, and for the treatment of ED. For example, inhibition of 3-KAT with trimetazidine, ranolazine or by genetically reducing 3-KAT synthesis, results in an increase in total body glucose uptake. Accordingly, inhibitors of fatty acid oxidation, such as a 3-KAT inhibitor or CPT-1 inhibitor, can be administered to treat hyperglycemia (e.g., as front line therapy). These agents are believed to reduce blood glucose levels by increasing uptake and oxidation of blood glucose.

Uptake of glucose by skeletal muscle and hepatic output of glucose (either by new glucose production from amino acids, called gluconeogenesis, or by hydrolysis of glycogen to d-glucose by a process called glycogenolysis) are considered the two most important regulators of plasma glucose homeostasis. Metformin, a biguanide antihyperglycemic drug that is not a 3-KAT inhibitor, is the only drug that has been demonstrated to reduce plasma glucose levels by reducing hepatic glucose output. (Inzucchi S E, et al., New Engl. J. Med. 338: 867-72 (1998)).

The methods, pharmaceutical compositions and kits of the invention provide benefits for the treatment of hyperglycemia (e.g., chronic hyperglycemia, hyperglycemia with diabetes, hyperglycemia without diabetes), impaired glucose tolerance, metabolic syndrome, T2DM, sequelae of hyperglycemia and/or T2DM including cardiovascular disease (e.g., leading to an increased prevalence of myocardial infarction, sudden death, acute coronary syndromes, stroke and chronic renal failure), diabetic retinopathy, diabetic nephropathy, diabetic neuropathy and ED. For example, as described herein, methods that comprise administering a 3-KAT inhibitor (e.g., trimetazidine) or other direct or indirect inhibitor of beta oxidation in combination with an agent that inhibits hepatic glucose production (e.g., metformin), target the two most important regulators of plasma glucose homeostasis. The effects of such agents (e.g., trimetazidine and metformin) on lowering blood glucose levels when administered as co-therapy can be additive or synergistic.

With respect to diabetic retinopathy, one drawback of therapeutic approaches that seek to inhibit neovascularization (e.g., that reduce VEGF) is that in the absence of neovascularization, the retina may remain hypoxic and degenerate. Advantageously, fatty acid oxidation inhibitors (such as trimetazidine) shift metabolism from fatty acid oxidation to glucose oxidation. This shift reduces the amount of oxygen required for the metabolic pathways to supply an adequate amount of energy to sustain normal cellular function. Thus, fatty acid oxidation inhibitors (such as trimetazidine) can compensate for hypoxia in the hypoxic retina. Similar benefits can be realized in treating diabetic nephropathy with fatty acid oxidation inhibitors (such as trimetazidine), because hypoxia and vascular inflammation contribute to glomerular injury. Thus, agents that reduce hypoxia, vascular inflammation and control blood sugar, such as trimetazidine, can provide a therapeutic benefit in restoring glomerular integrity to treat diabetic nephropathy.

As described herein, the pathology of diabetic neuropathy is multifactorial and involves both ischemic damage and sorbitol damage to nerve cells. Treatment with a fatty acid oxidation inhibitor, such as trimetazidine, increases the ratio of glucose to fatty acid oxidation, thus lowering the amount of oxygen required for an adequate supply of energy to sustain normal cellular function. Additionally, the drug-induced metabolic shift will increase the metabolism of glucose and sorbitol trapped intracellularly in the nerve cell. Although the inhibitor of fatty acid oxidation will not directly affect the conversion of glucose to sorbitol, glucose and sorbitol are in equilibrium and a shift to increased glucose oxidation will indirectly lower sorbitol levels. Additionally, an increase in glucose uptake and oxidation by other organs will reduce circulating glucose levels and hence reduce accumulation of intracellular glucose in nerve cells.

In one aspect, the invention relates to the use of an inhibiter of fatty acid oxidation (e.g., trimetazidine), another 3-KAT inhibitor (such as ranolazine) or other direct or indirect inhibitor of beta oxidation (such as a CPT-1 inhibitor, e.g., etomoxir) to increase glucose uptake and oxidation in skeletal muscle with the subsequent reduction in circulating plasma glucose levels as a means to treat hyperglycemia (e.g., chronic hyperglycemia). Hyperglycemia can be treated in this way in subjects with or without diabetes mellitus. For example, an inhibiter of fatty acid oxidation (e.g., trimetazidine), another 3-KAT inhibitor (such as ranolazine) or other direct or indirect inhibitor of beta oxidation (such as a CPT-1 inhibitor, e.g., etomoxir) can be administered to a subject in need thereof as front line therapy to increase glucose uptake and oxidation in skeletal muscle with the subsequent reduction in circulating plasma glucose levels, as a means to treat hyperglycemia (e.g., chronic hyperglycemia). A benefit of this therapeutic approach is that an increase in cGMP release is associated with administration of trimetazidine, other 3-KAT inhibitors (such as ranolazine) or other inhibitors of fatty acid oxidation. cGMP has vasodilatory effects which increase blood flow and the supply of glucose to tissues, thereby facilitating the uptake of glucose in skeletal muscle and all tissues.

In some embodiments, the invention is a method for treating type 2 diabetes mellitus comprising administering to a subject in need thereof a therapeutically effective amount of an inhibitor of fatty acid oxidation. In particular embodiments, the invention is a method for treating type 2 diabetes mellitus in a subject that does not have apparent cardiovascular disease (e.g., a subject that does not have impaired cardiac function, ischemic heart disease, cardiomyopathy, angina, or coronary artery disease) comprising administering to a subject in need thereof a therapeutically effective amount of an inhibitor of fatty acid oxidation. The method can be used as front line therapy for type 2 diabetes mellitus. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In preferred embodiments, the inhibitor of fatty acid oxidation is trimetazidine.

In some embodiments, the invention is a method for treating hyperglycemia (e.g., chronic hyperglycemia, hyperglycemia with diabetes, hyperglycemia without diabetes), impaired glucose tolerance and/or metabolic syndrome comprising administering to a subject in need thereof a therapeutically effective amount of an inhibitor of fatty acid oxidation. In particular embodiments, the invention is a method for treating hyperglycemia (e.g., chronic hyperglycemia, hyperglycemia with diabetes, hyperglycemia without diabetes), impaired glucose tolerance and/or metabolic syndrome in a subject that does not have apparent cardiovascular disease (e.g., a subject that does not have impaired cardiac function, ischemic heart disease, cardiomyopathy, angina, or coronary artery disease) comprising administering to a subject in need thereof a therapeutically effective amount of an inhibitor of fatty acid oxidation. The method can be used as front line therapy for treating hyperglycemia, impaired glucose tolerance and/or metabolic syndrome. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In preferred embodiments, the inhibitor of fatty acid oxidation is trimetazidine.

In another aspect, the invention relates to the use of trimetazidine, another 3-KAT inhibitor (such as ranolazine) or other direct or indirect inhibitor of beta oxidation (such as a CPT-1 inhibitor, e.g., etomoxir) in combination with an agent that inhibits hepatic glucose production (e.g., metformin). This combination targets the two most important regulators of plasma glucose homeostasis. This combination provides advantages over conventional therapy or therapy with one agent alone, because the activities of the agents, such as trimetazidine and metformin, are complementary. Trimetazidine blocks fatty acid metabolism (and increases glucose uptake and oxidation) which subsequently reduces lactate production, thus reducing the potential for the development of lactic acidosis (Monti, et al., Am. J. Physiol, Endocrinol. Metab. 209:54-59, (2006)), while metformin has been described as increasing the risk of developing lactic acidosis. In addition, the combination will provide cardioprotective effects that reduce cardiovascular risk and associated morbidity and mortality in T2DM patients. Therapy that effectively reduces blood glucose levels and also reduces cardiovascular risk factors is highly desirable. In fact, current therapies that effectively reduce blood glucose levels with high potency, such as sulfonylureas, are being abandoned by clinicians and patients because they do not reduce cardiovascular risk, mortality and morbidity associated with T2DM.

The combination of metformin and trimetazidine is also advantageous because these agents have similar plasma half-lives in humans and also have well-established dosing regimens for continuous/chronic use at 20 mg t.i.d. (three times a day) for trimetazidine and 500 mg t.i.d. for metformin (in their original formulations that were not designed to delay or prolong absorption kinetics). The combination of trimetazidine and metformin will increase glucose uptake and metabolism (oxidation) by all organs, and simultaneously reduce hepatic glucose output, while also reducing cardiovascular risk factors associated with hyperglycemia and T2DM. Thus, the effects of trimetazidine and metformin on lowering blood glucose levels when administered as co-therapy can be additive or synergistic.

In some embodiments, the invention is a method for treating type 2 diabetes mellitus comprising administering to a subject in need thereof a therapeutically effective amount of an inhibitor of fatty acid oxidation and an inhibitor of hepatic glucose output. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). Preferably, the inhibitor of hepatic glucose output is metformin. In preferred embodiments, the inhibitor of fatty acid oxidation is trimetazidine and the inhibitor of hepatic glucose output is metformin. In other embodiments, the inhibitor of fatty acid oxidation is ranolazine and the inhibitor of hepatic glucose output is metformin. In other embodiments, the inhibitor of fatty acid oxidation is etomoxir and the inhibitor of hepatic glucose output is metformin.

In some embodiments, the invention is a method for treating hyperglycemia (e.g., chronic hyperglycemia, hyperglycemia with diabetes, hyperglycemia without diabetes) and/or impaired glucose tolerance comprising administering to a subject in need thereof a therapeutically effective amount of an inhibitor of fatty acid oxidation and an inhibitor of hepatic glucose output. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). Preferably, the inhibitor of hepatic glucose output is metformin. In preferred embodiments, the inhibitor of fatty acid oxidation is trimetazidine and the inhibitor of hepatic glucose output is metformin. In other embodiments, the inhibitor of fatty acid oxidation is ranolazine and the inhibitor of hepatic glucose output is metformin. In other embodiments, the inhibitor of fatty acid oxidation is etomoxir and the inhibitor of hepatic glucose output is metformin.

In more particular embodiments, the invention is a method for treating type 2 diabetes mellitus comprising administering to a subject in need thereof trimetazidine and metformin, wherein trimetazidine is administered at about 30 mg/day to about 180 mg/day (e.g., about 61 mg/day to about 180 mg/day, about 90 mg/day, about 120 mg/day, about 150 mg/day, about 180 mg/day) and metformin is administered at about 1000 mg/day to about 2550 mg/day. The trimetazidine and metformin can be administered in any desired interval, for example, once a day, twice a day, three times a day or more often as desired. In one embodiment, trimetazidine is administered at about 20 mg t.i.d. and metformin is administered at about 500 mg t.i.d. In another embodiment, trimetazidine is administered at about 35 mg twice daily and metformin is administered at about 850 mg twice daily. In another embodiment, trimetazidine is administered at about 30 mg twice daily and metformin is administered at about 850 mg twice daily. In another embodiment, trimetazidine is administered at about 30 mg t.i.d., about 40 mg t.i.d., about 50 mg t.i.d., or about 60 mg t.i.d., and metformin is administered at about 1000 mg/day to about 2550 mg/day (e.g., 500 mg t.i.d. or 850 mg twice daily).

In other particular embodiments, the invention is a method for treating hyperglycemia (e.g., chronic hyperglycemia, hyperglycemia with diabetes, hyperglycemia without diabetes) impaired glucose tolerance and/or metabolic syndrome comprising administering to a subject in need thereof trimetazidine and metformin, wherein trimetazidine is administered at about 30 mg/day to about 180 mg/day (e.g., about 61 mg/day to about 180 mg/day, about 90 mg/day, about 120 mg/day, about 150 mg/day, about 180 mg/day) and metformin is administered at about 1000 mg/day to about 2550 mg/day. The trimetazidine and metformin can be administered in any desired interval, for example, once a day, twice a day, three times a day or more often as desired. In one embodiment, trimetazidine is administered at about 20 mg t.i.d. and metformin is administered at about 500 mg t.i.d. In another embodiment, trimetazidine is administered at about 35 mg twice daily and metformin is administered at about 850 mg twice daily. In another embodiment, trimetazidine is administered at about 30 mg t.i.d., about 40 mg t.i.d., about 50 mg t.i.d., or about 60 mg t.i.d., and metformin is administered at about 1000 mg/day to about 2550 mg/day (e.g., 500 mg t.i.d. or 850 mg twice daily).

In another aspect, the invention relates to the use of trimetazidine, another 3-KAT inhibitor (such as ranolazine) or other direct or indirect inhibitor of beta oxidation (such as a CPT-1 inhibitor, e.g., etomoxir) in combination with another antidiabetic drug. Suitable antidiabetic drugs for use in this combination include dipeptidyl peptidase IV inhibitors (e.g., sitagliptin, vildagliptin), sulfonylureas, disaccharidases (such as acarbose), amino acid insulin secretagogues (such as meglitinides), PPAR gamma agonists which include the thiazolidinediones (e.g, rosiglitazone, pioglitazone), incretin mimetics which include Byetta, and GLP-1 and GLP-1 analogs (such as exendin), insulin and insulin analogs (such as lispro and glargine), CPT-1 inhibitors (e.g., etomoxir), phosphodiesterase inhibitors, PPAR alpha receptor agonists, PPAR delta receptor agonists, dual PPAR alpha/gamma agonists, dual PPAR gamma/delta agonists, pan PPAR agonists, selective PPAR modulators, fatty acid analogs with affinity to the PPAR receptors, and inhibitors of beta oxidation.

In some embodiments, the invention is a method for treating type 2 diabetes mellitus comprising administering to a subject in need thereof a therapeutically effective amount of an inhibitor of fatty acid oxidation and another antidiabetic agent. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In some embodiments, the antidiabetic agent is a dipeptidyl peptidase IV inhibitor, such as sitagliptin or vildagliptin. In more particular embodiments, the inhibitor of fatty acid oxidation is trimetazidine, and the other antidiabetic agent is selected from the group consisting of sitagliptin, vildagliptin, acarbose, pioglitazone and rosiglitazone. In other particular embodiments, the inhibitor of fatty acid oxidation is ranolazine, and the other antidiabetic agent is selected from the group consisting of sitagliptin, vildagliptin, acarbose, pioglitazone and rosiglitazone. In other particular embodiments, the inhibitor of fatty acid oxidation is etomoxir, and the other antidiabetic agent is selected from the group consisting of sitagliptin, vildagliptin, acarbose, and rosiglitazone.

In some embodiments, the invention is a method for treating hyperglycemia (e.g., chronic hyperglycemia, hyperglycemia with diabetes, hyperglycemia without diabetes), impaired glucose tolerance and/or metabolic syndrome comprising administering to a subject in need thereof a therapeutically effective amount of an inhibitor of fatty acid oxidation and another antidiabetic agent. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In some embodiments, the antidiabetic agent is a dipeptidyl peptidase IV inhibitor, such as sitagliptin or vildagliptin. In more particular embodiments, the inhibitor of fatty acid oxidation is trimetazidine, and the other antidiabetic agent is selected from the group consisting of sitagliptin, vildagliptin, acarbose, pioglitazone and rosiglitazone. In other particular embodiments, the inhibitor of fatty acid oxidation is ranolazine, and the other antidiabetic agent is selected from the group consisting of sitagliptin, vildagliptin, acarbose, pioglitazone and rosiglitazone. In other particular embodiments, the inhibitor of fatty acid oxidation is etomoxir, and the other antidiabetic agent is selected from the group consisting of sitagliptin, vildagliptin, acarbose, pioglitazone and rosiglitazone.

The invention also relates to methods for treating sequelae of hyperglycemia and/or T2DM including T2DM-associated cardiovascular disease (e.g., leading to an increased prevalence of myocardial infarction, sudden death, acute coronary syndromes, stroke and chronic renal failure), diabetic retinopathy, diabetic nephropathy and/or diabetic neuropathy.

In some embodiments, the invention is a method for treating diabetic retinopathy, diabetic nephropathy and/or diabetic neuropathy comprising administering to a subject in need thereof a therapeutically effective amount of an inhibitor of fatty acid oxidation. In some embodiments, the diabetic retinopathy, diabetic nephropathy and/or diabetic neuropathy is in association with type 2 diabetes mellitus or hyperglycemia. In other embodiments, the diabetic retinopathy, diabetic nephropathy and/or diabetic neuropathy is not associated with diabetes mellitus. Diabetic nephropathy can be characterized by impaired glomerular filtration, microalbuminuria, proteinuria or any combination of the foregoing. In other particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In preferred embodiments, the inhibitor of fatty acid oxidation is trimetazidine.

In some embodiments, the invention is a method for treating diabetic retinopathy, diabetic nephropathy and/or diabetic neuropathy, comprising administering to a subject in need thereof a therapeutically effective amount of an inhibitor of fatty acid oxidation and an inhibitor of hepatic glucose output. In some embodiments, the diabetic retinopathy, diabetic nephropathy and/or diabetic neuropathy is in association with type 2 diabetes mellitus or hyperglycemia. In other embodiments, the diabetic retinopathy, diabetic nephropathy and/or diabetic neuropathy is not associated with diabetes mellitus. Diabetic nephropathy can be characterized by impaired glomerular filtration, microalbuminuria, proteinuria or any combination of the foregoing. In other particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). Preferably, the inhibitor of hepatic glucose output is metformin. In preferred embodiments, the inhibitor of fatty acid oxidation is trimetazidine and the inhibitor of hepatic glucose output is metformin. In other embodiments, the inhibitor of fatty acid oxidation is ranolazine and the inhibitor of hepatic glucose output is metformin. In other embodiments, the inhibitor of fatty acid oxidation is etomoxir and the inhibitor of hepatic glucose output is metformin.

In more particular embodiments, the invention is a method for treating diabetic retinopathy, diabetic nephropathy and/or diabetic neuropathy, comprising administering to a subject in need thereof trimetazidine and metformin, wherein trimetazidine is administered at about 30 mg/day to about 180 mg/day (e.g., about 61 mg/day to about 180 mg/day, about 90 mg/day, about 120 mg/day, about 150 mg/day, about 180 mg/day) and metformin is administered at about 1000 mg/day to about 2550 mg/day. The trimetazidine and metformin can be administered in any desired interval, for example, once a day, twice a day, three times a day or more often as desired. In one embodiment, trimetazidine is administered at about 20 mg t.i.d. and metformin is administered at about 500 mg t.i.d. In another embodiment, trimetazidine is administered at about 35 mg twice daily and metformin is administered at about 850 mg twice daily. In another embodiment, trimetazidine is administered at about 30 mg t.i.d., about 40 mg t.i.d., about 50 mg t.i.d., or about 60 mg t.i.d., and metformin is administered at about 1000 mg/day to about 2550 mg/day (e.g., 500 mg t.i.d. or 850 mg twice daily).

In some embodiments, the invention is a method for treating diabetic retinopathy, diabetic nephropathy and/or diabetic neuropathy, comprising administering to a subject in need thereof a therapeutically effective amount of an inhibitor of fatty acid oxidation and another antidiabetic agent. In some embodiments, the diabetic retinopathy, diabetic nephropathy and/or diabetic neuropathy is in association with type 2 diabetes mellitus or hyperglycemia. In other embodiments, the diabetic retinopathy, diabetic nephropathy and/or diabetic neuropathy is not associated with diabetes mellitus. Diabetic nephropathy can be characterized by impaired glomerular filtration, microalbuminuria, proteinuria or any combination of the foregoing. In other particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In some embodiments, the antidiabetic agent is a dipeptidyl peptidase IV inhibitor, such as sitagliptin or vildagliptin. In more particular embodiments, the inhibitor of fatty acid oxidation is trimetazidine, and the other antidiabetic agent is selected from the group consisting of sitagliptin, vildagliptin, acarbose, pioglitazone and rosiglitazone. In other particular embodiments, the inhibitor of fatty acid oxidation is ranolazine, and the other antidiabetic agent is selected from the group consisting of sitagliptin, vildagliptin, acarbose, pioglitazone and rosiglitazone. In other particular embodiments, the inhibitor of fatty acid oxidation is etomoxir, and the other antidiabetic agent is selected from the group consisting of sitagliptin, vildagliptin, acarbose, pioglitazone and rosiglitazone.

In another aspect, the invention relates to a pharmaceutical composition comprising an inhibitor of fatty acid oxidation (e.g., trimetazidine, another 3-KAT inhibitor (such as ranolazine) or other direct or indirect inhibitor of beta oxidation (such as a CPT-1 inhibitor, e.g., etomoxir)), an inhibitor of hepatic glucose output or another antidiabetic drug, and a physiologically acceptable carrier. The pharmaceutical composition can be a unit dose composition or a composition containing two or more doses. In certain embodiments, the pharmaceutical composition is a unit dose composition (e.g., a composition such as a tablet, capsule or liquid that is completely administered to the patient at one time). In some embodiments, the pharmaceutical composition is a unit dose composition (e.g., for dosing three times daily) comprising about 20 mg, about 30 mg, about 35 mg, about 40 mg, about 50 mg or about 60 mg of trimetazidine and about 500 mg of metformin. In other embodiments, the pharmaceutical composition is a unit dose composition (e.g., for dosing twice daily) comprising about 20 mg, about 35 mg, about 40 mg, about 50 mg or about 60 mg of trimetazidine and about 850 mg of metformin.

In particular embodiments, the invention is a pharmaceutical composition comprising trimetazidine and metformin and a physiologically acceptable carrier. In particular embodiments, the invention is a pharmaceutical composition comprising ranolazine and metformin and a physiologically acceptable carrier. In other particular embodiments, the invention is a pharmaceutical composition comprising etomoxir and metformin and a physiologically acceptable carrier. The composition can be a unit dose composition for administration once a day, twice a day, three times a day or more frequently. Preferably, the pharmaceutical composition (e.g., comprising trimetazidine and metformin) is for administration once a day or twice a day. Extended or slow release formulations can be prepared. For example, slow release formulations of metformin which is administered at a dose of 850 mg b.i.d. (twice a day) (GLUCOPHAGE XR (metformin hydrochloride extended-release tablets), Bristol-Myers Squibb Company) and of trimetazidine which is administered at a dose of 35 mg b.i.d. (twice daily) (VASTAREL MR, Servier) have been described.

The inventions also relates to a kit for treating hyperglycemia (e.g., chronic hyperglycemia, hyperglycemia with diabetes, hyperglycemia without diabetes), impaired glucose tolerance, metabolic syndrome, T2DM, and/or sequelae of hyperglycemia and/or T2DM including cardiovascular disease (e.g., leading to an increased prevalence of myocardial infarction, sudden death, acute coronary syndromes, stroke and chronic renal failure), diabetic retinopathy, diabetic nephropathy and diabetic neuropathy. The kit comprises separate pharmaceutical compositions (e.g., tablets, capsules, caplets). For example, the kit can contain a first pharmaceutical composition comprising an inhibitor of fatty acid oxidation, as described herein, and a second pharmaceutical composition comprising an inhibitor of hepatic glucose output or another antidiabetic agent, as described herein.

The kit preferably also includes a container for the separate pharmaceutical compositions, such as a bottle, a divided bottle, an envelope (e.g., of paper, foil or the like), a divided envelope, or a blister pack. The separate pharmaceutical compositions can be contained within the container so that they are not in contact with each other. For example, the first pharmaceutical composition and the second pharmaceutical composition can be in separate blisters in a blister pack. If desired, the separate pharmaceutical compositions can be in contact with each other in the container. For example, each blister in a blister pack can contain the first pharmaceutical composition and the second pharmaceutical composition.

In certain embodiments the kit comprises a first pharmaceutical composition comprising an inhibitor of fatty acid oxidation, such as a 3-KAT inhibitor (e.g., trimetazidine, ranolazine) or a CPT-1 inhibitor (e.g., etomoxir), and a second pharmaceutical composition comprising an inhibitor of hepatic glucose output (e.g., metformin). In particular embodiments, the kit comprises a first pharmaceutical composition comprising ranolazine and a second pharmaceutical composition comprising metformin. In particular embodiments, the kit comprises a first pharmaceutical composition comprising etomoxir and a second pharmaceutical composition comprising metformin.

In particular embodiments, the kit comprises a first pharmaceutical composition comprising trimetazidine and a second pharmaceutical composition comprising metformin. In some embodiments, the first pharmaceutical composition is a unit dose composition (e.g., for dosing three times daily) comprising about 20 mg, about 30 mg, about 35 mg, about 40 mg, about 50 mg or about 60 mg of trimetazidine, and the second pharmaceutical composition is a unit dose composition (e.g., for dosing three times daily) comprising about 500 mg of metformin. In other embodiments, the first pharmaceutical composition is a unit dose composition (e.g., for dosing twice daily) comprising about 20 mg, about 35 mg, about 40 mg, about 50 mg or about 60 mg of trimetazidine and the second pharmaceutical composition is a unit dose composition (e.g., for dosing twice daily) comprising about 850 mg of metformin.

In other embodiments the kit comprises a first pharmaceutical composition comprising an inhibitor of fatty acid oxidation, such as a 3-KAT inhibitor (e.g., trimetazidine, ranolazine) or a CPT-1 inhibitor (e.g., etomoxir), and a second pharmaceutical composition comprising another antidiabetic agent, as described herein (e.g., a dipeptidyl peptidase IV inhibitor). In particular embodiments, the kit comprises a first pharmaceutical composition comprising trimetazidine and a second pharmaceutical composition comprising sitagliptin, vildagliptin, acarbose, pioglitazone, or rosiglitazone. In particular embodiments, the kit comprises a first pharmaceutical composition comprising ranolazine and a second pharmaceutical composition comprising sitagliptin, vildagliptin, acarbose, pioglitazone, or rosiglitazone. In other particular embodiments, the kit comprises a first pharmaceutical composition comprising etomoxir and a second pharmaceutical composition comprising sitagliptin, vildagliptin, acarbose, pioglitazone, or rosiglitazone.

In another aspect, the invention relates to the use of an inhibitor of fatty acid oxidation (e.g., trimetazidine, another 3-KAT inhibitor (such as ranolazine) or other direct or indirect inhibitor of beta oxidation (such as a CPT-1 inhibitor, e.g., etomoxir)) to induce the production of cGMP in a subject. Inducing cGMP produces a vasodilatory effect that can be used to increase perfusion or to treat ED.

In some embodiments, the invention is a method for treating ED comprising administering to a subject in need thereof a therapeutically effective amount of an inhibitor of fatty acid oxidation. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In preferred embodiments, the inhibitor of fatty acid oxidation is trimetazidine.

The invention also relates to the use of an inhibitor of fatty acid oxidation (e.g., trimetazidine, another 3-KAT inhibitor (such as ranolazine) or other direct or indirect inhibitor of beta oxidation (such as a CPT-1 inhibitor, e.g., etomoxir)) in combination with an agent that inhibits cGMP degradation (e.g., a phosphodiesterase inhibitor, such as a phosphodiesterase 5 inhibitor). This combination targets the important regulators of erectile function by increasing the amount of erection-inducing cGMP, and inhibiting degradation of cGMP. Thus, administering trimetazidine or another inhibitor of fatty acid oxidation (e.g., another 3-KAT inhibitor or a CPT-1 inhibitor) and a phosphodiesterase inhibitor (e.g., a phosphodiesterase 5 inhibitor such as tadalafil, sildenafil or vardenafil) can have additive or synergistic effects on erectile function.

In some embodiments, the invention is a method for treating ED comprising administering to a subject in need thereof a therapeutically effective amount of an inhibitor of fatty acid oxidation and an inhibitor of cGMP degradation. In particular embodiments, the inhibitor of fatty acid oxidation is a 3-KAT inhibitor (e.g., trimetazidine, ranolazine). In other embodiments, the inhibitor of fatty acid oxidation is a CPT-1 inhibitor (e.g., etomoxir). In preferred embodiments, the inhibitor of fatty acid oxidation is trimetazidine. In other preferred embodiments, the inhibitor of cGMP degradation is a phosphodiesterase 5 inhibitor. In particularly preferred embodiments, the method of treating ED comprises administering a therapeutically effective amount of trimetazidine and a phosphodiesterase 5 inhibitor selected from the group consisting of tadalafil, sildenafil and vardenafil. In other embodiments, the method of treating ED comprises administering a therapeutically effective amount of ranolazine and a phosphodiesterase 5 inhibitor selected from the group consisting of tadalafil, sildenafil and vardenafil. In other embodiments, the method of treating ED comprises administering a therapeutically effective amount of etomoxir and a phosphodiesterase 5 inhibitor selected from the group consisting of tadalafil, sildenafil and vardenafil.

In another aspect, the invention relates to a pharmaceutical composition comprising an inhibitor of fatty acid oxidation (e.g., trimetazidine, another 3-KAT inhibitor (such as ranolazine) or other direct or indirect inhibitor of beta oxidation (such as a CPT-1 inhibitor, e.g., etomoxir)), an inhibitor of cGMP degradation, and a physiologically acceptable carrier. The pharmaceutical composition can be a unit dose composition or a composition containing two or more doses. In certain embodiments, the pharmaceutical composition is a unit dose composition (e.g., a composition such as a tablet, capsule or liquid that is completely administered to the patient at one time).

In particular embodiments, the invention is a pharmaceutical composition comprising trimetazidine, a phosphodiesterase 5 inhibitor (e.g., tadalafil, sildenafil, vardenafil) and a physiologically acceptable carrier. In particular embodiments, the invention is a pharmaceutical composition comprising ranolazine, a phosphodiesterase 5 inhibitor (e.g., tadalafil, sildenafil, vardenafil) and a physiologically acceptable carrier. In particular embodiments, the invention is a pharmaceutical composition comprising etomoxir, a phosphodiesterase 5 inhibitor (e.g., tadalafil, sildenafil, vardenafil) and a physiologically acceptable carrier. The compositions are preferably for at will dosing, or once a day dosing.

The inventions also relates to a kit for treating ED. The kit comprises separate pharmaceutical compositions (e.g., tablets, capsules, caplets). For example, the kit can contain a first pharmaceutical composition comprising an inhibitor of fatty acid oxidation, as described herein, and a second pharmaceutical composition comprising an agent that inhibits cGMP degradation (e.g., a phosphodiesterase inhibitor, such as a phosphodiesterase 5 inhibitor).

The kit preferably also includes a container for the separate pharmaceutical compositions, such as a bottle, a divided bottle, an envelope (e.g., of paper, foil or the like), a divided envelope, or a blister pack. The separate pharmaceutical compositions can be contained within the container so that they are not in contact with each other. For example, the first pharmaceutical composition and the second pharmaceutical composition can be in separate blisters in a blister pack. If desired, the separate pharmaceutical compositions can be in contact with each other in the container. For example, each blister in a blister pack can contain the first pharmaceutical composition and the second pharmaceutical composition.

In certain embodiments the kit comprises a first pharmaceutical composition comprising an inhibitor of fatty acid oxidation, such as a 3-KAT inhibitor (e.g., trimetazidine, ranolazine) or a CPT-1 inhibitor (e.g., etomoxir), and a second pharmaceutical composition comprising an agent that inhibits cGMP degradation (e.g., a phosphodiesterase inhibitor, such as a phosphodiesterase 5 inhibitor). In particular embodiments, the kit comprises a first pharmaceutical composition comprising trimetazidine and a second pharmaceutical composition comprising a phosphodiesterase 5 inhibitor (e.g., tadalafil, sildenafil, vardenafil). In particular embodiments, the kit comprises a first pharmaceutical composition comprising ranolazine and a second pharmaceutical composition comprising a phosphodiesterase 5 inhibitor (e.g., tadalafil, sildenafil, vardenafil). In particular embodiments, the kit comprises a first pharmaceutical composition comprising etomoxir and a second pharmaceutical composition comprising a phosphodiesterase 5 inhibitor (e.g., tadalafil, sildenafil, vardenafil).

In some embodiments of the kit, the first pharmaceutical composition is a unit dose composition (e.g., for at will dosing or once daily dosing) comprising about 20-180 mg of trimetazidine, and the second pharmaceutical composition is a unit dose composition (e.g., for at will dosing or once daily dosing) comprising a phosphodiesterase 5 inhibitor (e.g., about 20 mg of tadalafil, about 50 mg or sildenafil, about 10 mg of vardenafil).

A “subject” is preferably a mammal, such as a human (Homo sapiens), but can also be an animal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

A “therapeutically effective amount” is an amount sufficient to achieve a desired therapeutic effect, such as an amount that results in a reduction of glycated hemoglobin (HbA1c), for example, about 0.5%, about 1%, about 1.5%, about 2%, about 2.5%, about 3% or greater reduction. For monotherapy, a reduction of HbA1c of about 0.5% to about 1.5% is considered therapeutically effective. For co-therapy, a reduction of HbA1c of about 1% to about 2.5% or more is preferred. For treating ED, a therapeutically effective amount is an amount that allows the subject to achieve an erection sufficient for satisfactory sexual activity. This can be assessed using the International Index of Erectile Function assessment, that is based on a questionnaire which produces a score of up to 30 points. Higher scores reflect better erectile function. Generally an increase in the erectile function score of about 4 or more points is considered effective. Effectiveness can also be assessed using questions 2 and 3 of the Sexual Encounter Profile. Question 2 is whether the subject was able to insert his penis into his partner's vagina, and question 3 is whether the subject's erection lasted long enough for him to have successful intercourse. An increase of affirmative answers to question 2 and question 3 of about 15% is generally considered effective.

The amount of compound administered to the subject will depend on the type and severity of the disease and on the characteristics of the individual, such as general health, age, sex, body weight and tolerance to drugs. It will also depend on the degree, severity and type of disease. The skilled clinician will be able to determine appropriate dosages depending on these and other factors.

When trimetazidine is administered to treat type 2 diabetes mellitus, hyperglycemia, impaired glucose tolerance, metabolic syndrome or T2DM-associated diseases it can be administered at about 30 mg/day to about 180 mg/day (e.g., about 61 mg/day to about 180 mg/day, about 90 mg/day, about 120 mg/day, about 150 mg/day, about 180 mg/day). The trimetazidine can be administered in any desired interval, for example, once a day, twice a day, three times a day or more often as desired. When trimetazidine is administered as front line therapy, for example for hyperglycemia or T2DM, it is preferably administered at about 61 mg/day to about 180 mg/day. For example, trimetazidine can be administered at about 30 mg t.i.d., about 40 mg t.i.d., about 50 mg t.i.d., or about 60 mg t.i.d.

When compounds are administered in combination, the compounds can be administered in a manner that afford overlap of pharmacological activity, for example, concurrently or sequentially. The compounds can be administered by any suitable route, including, for example, orally (e.g., in capsules, suspensions or tablets), by inhalation (e.g., intrabronchial, intranasal, oral inhalation or intranasal drops), or by parenteral administration. Parenteral administration can include, for example, systemic administration, such as by intramuscular, intravenous, subcutaneous or intraperitoneal injection. The compound can also be administered transdermally, topically, or rectally, depending on the disease or condition to be treated. Oral administration is the preferred mode of administration. The compound can be administered to the individual as part of a pharmaceutical or physiological composition.

The compounds can be prepared using known methods and can be prepared and administered as neutral compounds or salts. As used herein, “pharmaceutically or physiologically acceptable salts” are those salts (e.g., carboxylate salts, amino acid addition salts) of compounds that are suitable for use in contact with the tissues of a subject without undue toxicity, irritation, allergic response, and the like, commensurate with a reasonable benefit/risk ratio, and effective for their intended use, as well as the zwitterionic forms, where possible, of the compounds of the invention.

Pharmaceutically or physiologically acceptable acid addition salts of the compounds described herein include salts derived from nontoxic inorganic acids such as hydrochloric, nitric, phosphoric, sulfuric, hydrobromic, hydroiodic, hydrofluoric, phosphorous, and the like, and salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, and the like. Such acid addition salts include, for example, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate and methanesulfonate salts. Also contemplated are salts of amino acids such as arginate, gluconate, galacturonate and the like. (See, e.g., Berge S. M., et al., “Pharmaceutical Salts,” J. Pharma. Sci., 66:1 (1977)).

Acid addition salts of compounds that contain a basic group (e.g., amine) can be prepared using suitable methods. For example, acid addition salts can be prepared by contacting the free base form of a compound with a sufficient amount of a desired acid to produce the salt in the conventional manner. The free base form can be regenerated by contacting the salt form with a base and isolating the free base in the conventional manner. The free base form of a compound can differ somewhat from salt forms in certain physical properties such as solubility in polar solvents.

Pharmaceutically or physiologically acceptable base addition salts can be formed with suitable metals or amines, such as alkali and alkaline earth metals or organic amines. Examples of metals that are suitable for use as cations in base addition salts include sodium, potassium, magnesium, calcium and the like. Amines suitable for use as cations in base addition salts include N,N-dibenzylethylenediamine, chloroprocaine, choline, diethanolamine, dicyclohexylamine, ethylenediamine, N-methylglucamine, and procaine. (See, e.g., Berge S. M. et al., “Pharmaceutical Salts,” J. Pharma. Sci., 66:1 (1977)).

Base addition salts of compounds which contain an acidic group (e.g., carboxylic acid) can be prepared using suitable methods. For example, the free acid form of a compound can be contacted with a sufficient amount of the desired base to produce a salt in the conventional manner. The free acid form can be regenerated by contacting the salt form with a suitable acid and isolating the free acid in the conventional manner. The free acid form of a compound can differ somewhat from salt forms in certain physical properties such as solubility in polar solvents.

Preferred salts of certain compounds are: trimetazidine dihydrochloride, etomoxir sodium hydrate, metformin hydrochloride, rosiglitazone maleate, sitagliptin phosphate, sildenafil citrate, and vardenafil HCl.

The invention also relates to pharmaceutical and/or physiological compositions which contain one or more of the compounds described herein. Such compositions can be formulated for administration by any desired route, such as orally, topically, by inhalation (e.g., intrabronchial, intranasal, oral inhalation or intranasal drops), rectally, transdermally, or parenterally. Generally, the compositions comprise a compound described herein (i.e., one or more compounds) as the active ingredient and a (one or more) suitable carrier, diluent, excipient, adjuvant and/or preservative. Formulation of a compound will vary according to the route of administration selected (e.g., solution, emulsion, capsule). Standard pharmaceutical formulation techniques can be employed. See generally, “Remington's Pharmaceutical Science,” 18^{th} Edition, Mack Publishing. (1990); and Baker, et al., “Controlled Release of Biological Active Agents,” John Wiley and Sons (1986), the entire teachings of both of the foregoing are incorporated herein by reference.

The presence of microorganisms in the compositions can be controlled by various antibacterial and/or antifungal agents, for example, parabens, chlorobutanol, alcohols (e.g., phenol, benzyl alcohol), sorbic acid, and the like. It may also be desirable to include isotonic agents, for example sugars, sodium chloride, and the like.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, excipients or vehicles include physiological saline, phosphate-buffered saline, Hank's solution, Ringer's-lactate and the like, ethanol, polyols (propyleneglycol, polyethyleneglycol, glycerol, and the like), vegetable oils (such as olive oil) and injectable organic esters such as ethyl oleate, or any suitable mixture thereof. Fluidity can be adjusted, for example, by the use of a coating such as lecithin, by the maintenance of the required particle size in the case of dispersions and by the use of surfactants. When prolonged absorption of an injectable pharmaceutical composition is desired, agents that delay absorption, for example, aluminum monostearate and gelatin can be included.

Solid dosage forms for oral administration include, for example, capsules, tablets, pills, powders, and granules. In such solid dosage forms, the active ingredient (i.e., one or more compounds) can be admixed with one or more carrier or excipient such as sodium citrate or dicalcium phosphate; (a) fillers or extenders, for example, starches, lactose, sucrose, glucose, mannitol, silicic acid, polyethyleneglycols, and the like; (b) binders, for example, carboxymethylcellulose, alignates, gelatin, polyvinylpyrrolidone, sucrose, and acacia; (c) humectants, for example, glycerol; (d) disintegrating agents, for example, agar-agar, calcium carbonate, potato or tapioca starch, alginic acid, certain complex silicates, and sodium carbonate; (e) solution retarders, for example paraffin; (f) absorption accelerators, for example, quaternary ammonium compounds; (g) wetting agents, for example, cetyl alcohol, and glycerol monostearate; (h) adsorbents, for example, kaolin and bentonite; and (i) lubricants, for example, talc, calcium stearate, magnesium stearate, solid polyethylene glycols, sodium lauryl sulfate, or mixtures thereof. Solid compositions, such as those for oral administration, can also comprise buffering agents. Such solid compositions or solid compositions that are similar to those described can be provided in soft-filled or hard-filled gelatin capsules if desired.

Solid dosage forms such as tablets, capsules, pills, and granules can be prepared with coatings and shells, such as enteric coatings or other suitable coatings or shells. Several such coating and/or shells are well known in the art, and can contain opacifying agents, and can also be of such composition that they release the active compound or compounds in a certain part of the intestinal tract in a delayed manner. Examples of embedding compositions which can be used are polymeric substances and waxes. The active compounds can also be used in microencapsulated form, if appropriate, with, for example, one or more of the above-mentioned carriers or excipients.

Liquid dosage forms for oral administration include pharmaceutically acceptable emulsions, solutions, suspensions, syrups, and elixirs. In addition to the active compounds, the liquid dosage forms can contain a suitable carrier or excipient, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like. If desired, the composition can also include wetting agents, emulsifying agents, suspending agents, sweetening, flavoring and/or perfuming agents. Suspensions can contain suspending agents, such as, ethoxylated isostearyl alcohols, polyoxyethylene sorbitol and sorbitan esters, microcrystalline cellulose, aluminum metahydroxide, bentonite, agar-agar, tragacanth, and the like. Mixtures of suspending agents can be employed if desired. Suppositories (e.g., for rectal or vaginal administration) can be prepared by mixing one or more compounds with suitable nonirritating excipients or carriers such as cocoa butter, polyethyleneglycol, or a suppository wax which is solid at room temperature but liquid at body temperature and melts in the rectum or vagina, thereby releasing the active ingredient.

Dosage forms for topical administration include ointments, powders, sprays and inhalants. The active ingredient can be admixed under suitable conditions (e.g., sterile conditions) with a physiologically acceptable carrier and any preservatives, buffers, or propellants as may be required. Ophthalmic formulations, eye ointments, powders, and solutions can also be prepared, for example, using suitable carriers or excipients. For inhalation, the compound can be solubilized and loaded into a suitable dispenser for administration (e.g.; an atomizer, nebulizer or pressurized aerosol dispenser).

The quantity of active ingredient (one or more compounds of the invention) in the composition can range from about 0.1% to about 99.9% by weight. Preferably the quantity of active ingredient is about 10% to about 90%, or about 20% to about 80% by weight. A unit dose preparation can contain from 1 mg to about 2000 mg active ingredient. Preferably a unit dose formulation comprises about 20 mg to about 40 mg of trimetazidine and about 500 mg to about 1000 mg, or about 750 mg to about 850 mg of metformin.

The invention also relates to a method for treating a patient in need thereof for metabolic syndrome or diabetes and endothelial dysfunction comprising administering a combination of two or more compounds selected from the group consisting of an HMG CoA reductase inhibitor, a partial fatty acid oxidation (“pFox”) inhibitor, one or more oral hypoglycemics, a protein kinase C inhibitor, and an acetyl-CoA carboxylase inhibitor. This aspect of the invention is also the subject of disclosure and claims in U.S. patent application Ser. No. 11/373,658 (US 2006/0205727).

In some embodiments, the patient has type II diabetes. Alternatively or additionally, the patient can have coronary heart disease, atherosclerotic vascular disease, congestive heart failure, peripheral arterial disease and claudication, chronic angina, unstable angina, microvascular angina due to left ventricle hypertrophy, microvascular angina, or three or more risk factors for metabolic syndrome selected from the group consisting of abdominal obesity, elevated blood pressure, atherogenic dyslipidemia (high triglycerides, low HDL and small, dense LDL), impaired fasting glucose or glucose intolerance, proinflammatory state and prothrombotic state.

In some aspects, the invention is in the field of treating endothelial dysfunction, angina and diabetes, especially through the use of a combination of a partial fatty acid oxidation (“pFox”) inhibitor, such as trimetazidine, an HMG CoA reductase inhibitor (“statin”), one or more oral hypoglycemic compounds, protein kinase C inhibitors, and acetyl-CoA carboxylase inhibitors.

In some embodiments, the patient has type II diabetes. Alternatively or additionally, the patient can have coronary heart disease, atherosclerotic vascular disease, congestive heart failure, peripheral arterial disease and claudication, chronic angina, unstable angina, microvascular angina due to left ventricle hypertrophy, microvascular angina, or three or more risk factors for metabolic syndrome selected from the group consisting of abdominal obesity, elevated blood pressure, atherogenic dyslipidemia (high triglycerides, low HDL and small, dense LDL), impaired fasting glucose or glucose intolerance, proinflammatory state and prothrombotic state.

In another aspect, the invention is in the field of treating endothelial dysfunction, angina and diabetes, especially through the use of a combination which comprises a partial fatty acid oxidation inhibitor and a compound selected from the group consisting of a protein kinase C inhibitor and an acetyl-CoA carboxylase inhibitor.

The combination of a HMG CoA reductase inhibitor like a statin, such as simvastatin, with a pFox inhibitor such as trimetazidine (“Simetazidine”), is particularly advantageous for treatment of end-stage complications, such as acute coronary syndrome (ACS) and chronic angina, especially in type II diabetics. The combination therapy is also useful in the treatment and/or prevention of chronic heart failure (CHF) and peripheral arterial disease (PAD). A nitric oxide agonist, nitric oxide generator or an upregulator of nitric oxide synthase can also be administered or a pFOX inhibitor or HMG CoA reductase inhibitor having such an activity can also be administered. The combination of a nitric oxide (NO) mechanism that results in increased NO production with pFox inhibition simultaneously treats both the effect and the cause of angina. One or more oral hypoglycemic compounds such as biguanides, insulin sensitizers, such as thiazolidinediones, alpha-glucosidase inhibitors, insulin secretagogues, and dipeptidyl peptidase IV inhibitors, protein kinase C (PKC) inhibitors, and acetyl-CoA carboxylase inhibitors can also be used in combination with the HMG CoA reductase inhibitors and/or pFox inhibitors, especially in type II diabetics, to control glucose levels and treat endothelial dysfunction. The drugs can be given in combination (e.g. a single tablet) or in separate dosage forms, administered simultaneously or sequentially. In the preferred form the statin is given in a dose of between 5 and 80 mg/day in two separate doses, and the pFox inhibitor is administered in a sustained or extended dosage formulation at a dose of 20 mg three times a day or 35 mg two times a day. The dose of the oral hypoglycemic, PKC inhibitor, or acetyl-CoA carboxylase inhibitor varies with the type of drug used.

A combination therapy has been designed to provide the benefits of treatment with a trimetazidine or other pFox inhibitor in combination with an HMG CoA reductase inhibitor, such as a statin. One or more oral hypoglycemics, including biguanides, insulin sensitizers, alpha-glucosidase inhibitors, insulin secretagogues, may also be used in combination with the HMG CoA reductase inhibitor and pFox inhibitor for the treatment of diabetes and endothelial dysfunction. In addition, dipeptidyl peptidase IV inhibitors, which are also hypoglycemics, protein kinase C inhibitors, acetyl-CoA carboxylase inhibitors, or selective rho-kinase inhibitors may be used in combination with the HMG CoA reductase inhibitor and/or pFox inhibitor.

A “pFox inhibitor” is any compound that shifts myocardial substrate utilization from free fatty acid to glucose, regardless of the enzyme inhibited. A pFox inhibitor, most preferably one which does not prolong QT intervals, can be used in combination with a HMG CoA reductase inhibitor, common referred to as “statins”, and optionally an oral hypoglycemic for the treatment of endothelial dysfunction and diabetes. The combination of a pFox inhibitor with an HMG CoA reductase inhibitor has a dual mechanism of both reversing endothelial dysfunction through the nitric oxide pathway and reducing ischemia thereby relieving angina and improving long term outcome.

The piperazine derivatives ranolazine and trimetazidine are examples of pFox inhibitors whose mechanism of action involves shifting ATP production away from fatty acid oxidation in favor of glucose oxidation. Inhibition of fatty acid oxidation results in a reduction in the inhibition of pyruvate dehydrogenase and an increase in glucose oxidation. The amount of oxygen required to phosphorylate a given amount of ATP is greater during fatty acid oxidation than during carbohydrate oxidation. Thus, increasing glucose oxidation reduces oxygen demand without decreasing the ability of tissue to do work. Trimetazidine has also been shown to: (1) reduce the levels of plasma C-reactive protein in the course of acute myocardial infarction treated with streptokinase and intravenous trimetazidine infusion (Blaha, et al., Acta Medica, 44(4), 135-40 (2001)); (2) have a beneficial effect in patients with circulatory deficiency through the improvement of hemostatic and biochemical parameters (Demidova, et al., Ter. Arkh., 70(6), 41-44 (1998)); and (3) induce functional improvement in patients with dilated cardiomyopathy via significant improvement of left ventricular function (Barsotti, et al., Heart, 91(2), 161-165 (2005)). Clinical results also suggest that the inflammatory response was limited in patients treated with trimetazidine (Barostti, et al.).

Ranolazine and trimetazidine are described in U.S. Pat. Nos. 4,567,264, and 4,663,325, respectively. Ranolazine is not preferred because it causes QT interval prolongation and undergoes metabolism via the CYP3A4 system in the liver and is prone to drug-drug interactions which further aggravate QT interval prolongation. Other suitable pFOX inhibitors include perhexiline maleate and mildronate.

Perhexiline maleate is an anti-anginal agent. Its mechanism of action as an anti-anginal agent has not been fully elucidated in humans; however, in vitro studies suggest that perhexiline causes inhibition of myocardial fatty acid catabolism (e.g. by inhibition of carnitine palmitoyltransferase-1: CPT-1) with a concomitant increase in glucose utilization and consequent oxygen-sparing effect. This is likely to have two consequences: (i) increased myocardial efficiency, and (ii) decreased potential for impairment of myocardial function during ischemia.

The inhibition of CPT-1 is likely to contribute to the anti-ischaemic effects of perhexiline. Animal studies indicate a direct action of the medicine on the myocardium dependent in part on the marked degree of tissue binding. In vitro studies indicate a non-specific depressant effect of perhexiline on all smooth muscle. It also inhibits the spontaneous depolarisation of Purkinje fibers in the dog myocardium and reduces sodium and potassium conductance. The dosage range of perhexiline is typically 100 mg to 300 mg daily; however, dosages of 400 mg per day may be required. Perhexiline maleate is commercially available in 100 mg tablets.

Mildronate ameliorates cardiac function during ischemia by modulating myocardial energy metabolism. Biochemical and pharmacological evidence suggests that the mechanism of action of mildronate is based on the regulatory effect on carnitine concentration, whereby mildronate treatment shifts the myocardial energy metabolism from fatty acid oxidation to the more favorable glucose oxidation under ischemic conditions (Dambrova, et al., Trends in Cardiovascular Medicine, Vol. 12, No. 6 (2002)). The dosage range for mildronate is typically between 500 mg and 1000 mg daily, in divided doses. Mildronate is commercially available in 250 mg and 500 mg capsules as well as a 10% injectable solution and a syrup.

Statins

There are a number of statins that are available and approved for use. These include mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin. The preferred statins include pravastatin, torvastatin, fluvastatin, lovastatin, and metastatin. The statin compounds are administered in regimens and at dosages known in the art. For instance, Cervistatin, which is sold by Bayer Corporation as Baycol™, has a recommended dosage of 0.3 mg once daily in the evening, with a starting dose for patients with significant renal failure of 0.2 mg per day, taken once daily in the evening. Fluvastatin sodium, marketed by Novartis Pharmaceuticals as Lescol™, is recommended for a 20-80 mg daily oral dose range, preferably between 20 and 40 mg/day for the majority of patients. 20 to 40 mg daily doses are preferably taken once daily at bedtime. 80 mg daily doses is prescribed as 40 mg doses b.i.d. and recommended only for those individuals in which the 40 mg daily dose is inadequate to lower LDL levels satisfactorily. Atorvastatin, offered by Parke Davis as Lipitor™, has a recommended starting daily dose of 10 mg once daily, with an overall daily dose range of from 10 to 80 mg. Simvastatin, marketed by Merck & Co., Inc., may be administered with a starting dose of 20 mg once a day in the evening, or a 10 mg dose per day for those requiring only a moderate reduction in LDL levels. The recommended overall daily dosage range taken as a single evening dose is from 5 to 80 mg. Pravastatin sodium, sold as Pravachol™ by Bristol-Meyers Squibb, has a recommended starting dose of 10 or 20 mg per day, taken daily as a single dose at bedtime, with a final overall daily range of from 10 to 40 mg. Lovastatin, sold by Merck & Co. as Mevacor™, has a recommended daily starting dosage of 20 mg per day taken with the evening meal. The recommended final daily dosage range is from 10 to 80 mg per day in single or divided doses.

HMG CoA reductase inhibitors have been shown to lower blood cholesterol levels by upregulating lipoprotein clearance receptors in the liver (Brown & Goldstein, Science 232, 34-47 (1986)). Based on the Heart Protection Study and the A to Z trial the preferred simvastatin dose should be 40 mg total/day. This could be formulated, for example, as 20 mg simvastatin immediate release combined with 35 mg of the new trimetazidine MR for BID dosing or it could be 13.33 mg simvastatin/20 mg immediate release trimetazidine for TID dosing. In April 2004, the US Food and Drug administration approved the use of simvastatin for treating existing coronary heart disease and diabetes irrespective of cholesterol levels. This was based on the results of the Heart Protection Study, a seven year, 22,000 patient study which showed benefits regardless of the levels of cholesterol of the individuals in the trial. In the PROVE-IT trial some benefits were seen in the treatment of acute coronary syndrome in the first 30 days of the trial with 80 mg/day of atorvastatin that was believed to be unrelated to cholesterol lowering.

Nitric Oxide Agonists/Generators/Upregulators of Nitric Oxide Synthase

In one embodiment, a nitric oxide agonist, nitric oxide generator or an upregulator of nitric oxide synthase is given in combination with an HMG CoA reductase inhibitor and a partial fatty acid oxidation (“pFox”) inhibitor. Suitable nitric oxide agonists or upregulators of nitric oxide synthase include angiotensin II receptor blockers (ARB's), angiotensin converting enzyme (ACE) inhibitors, endothelial nitric oxide synthase agonists, peroxisome proliferator-activated receptor activators, and cilostazol.

Angiotensin-II receptor antagonists (or blockers) are selective for the angiotensin II (type 1 receptor). Examples of angiotensin-II receptor antagonists are losartan (Cozaar) (50-200 mg/day), valsartan (Diovan) (80 to 320 mg), irbesartan (Avapro) (75-300 mg/day), candesartan (Atacand) (8-64 mg/day) and telmisartan (Micardis) (40-160 mg/day). Other angiotensin-II receptor antagonists currently under investigation include eprosartan, tasosartan and zolarsartan.

Angiotensin Converting Enzyme (ACE) Inhibitors generate nitric oxide in the wall of small arteries. Suitable angiotensin-converting enzyme inhibitors along with recommended daily doses, include, but are not limited to, alacepril, benazepril (10-80 mg/day), captopril (25-450 mg/day), ceranapril, cilazapril, delapril, duinapril, enalapril (5-40 mg/day), enalaprilat, fosinopril (10-80 mg/day), imidapril, lisinopril (10-40 mg/day), moexipril (7.5-30 mg/day), moveltipril, pentopril, perindopril (4-16 mg/day), quinapril (10-80 mg/day), ramipril (2.5-20 mg/day), rentipril, spirapril, temocapril, trandolapril (1-8 mg/day), and zofenopril. The angiotensin-converting enzyme inhibitors are described more fully in the literature, such as in Goodman & Gilman, The Pharmacological Basis of Therapeutics (9th Edition), McGraw-Hill, 1995; and the Merck Index on CD-ROM, Twelfth Edition.

There are a number of compounds that are known to upregulate eNOS expression and/or increase eNOS activity. These are described in U.S. Patent Publication No. 20040254238 and U.S. Pat. No. 6,425,881, and include acetylcholine, cyclosporin A, FK506, felodipine, nicorandil, nifedipine, diltiazem, resveritrol, sapogrelate, quinapril and nebivolol. The combination of nebivolol and a pFox inhibitor, such as trimetazidine, should be beneficial for the treatment of angina and hypertension. Statins are also known activators of eNOS. For example, high density lipoprotein (“HDL”) causes potent stimulation of eNOS activity through binding to SR-BI. Statins, such as simvastatin and atorvastatin increase the concentration of HDL (atorvastatin more so than simvastatin). Mixtures of NO donors may also have this effect as described in U.S. Pat. No. 5,543,430 which describes nitroglycerin as an eNOS agonist in combination with arginine.

In humans, peroxisome proliferator-activated receptors (PPARs) are found in key target tissues for insulin action such as adipose tissue, skeletal muscle, and liver. Activation of PPARgamma. nuclear receptors regulates the transcription of insulin-responsive genes involved in the control of glucose production, transport, and utilization. In addition, PPARgamma-responsive genes also participate in the regulation of fatty acid metabolism. Suitable peroxisome proliferator-activated receptor activators include those agents that bind to the peroxisome proliferator-activated receptor gamma (PPAR-gamma). Examples of such compounds include the thiazolidinediones, troglitazone (Rezulin), rosiglitazone (Avandia) and pioglitazone (Actos), which are described below.

Cilostazol (6-[4-(1-cyclohexyl-1H-tetrazol-5-yl)butoxy]-3,4-dihydro-2(1H)-quinolinon-e, a treatment for intermittent claudication, is sold as PLETAL™ Otsuka America Pharmaceutical. Intermittent claudication is a condition caused by narrowing of the arteries that supply the legs with blood. Patients with intermittent claudication develop pain when they walk because not enough oxygen-containing blood reaches the active leg muscles. Cilostazol reduces the pain of intermittent claudication by dilating the arteries, thereby improving the flow of blood and oxygen to the legs. Cilostazol and some of its metabolites are cyclic AMP (cAMP) phosphodiesterase III inhibitors (PDE III inhibitors), inhibiting phosphodiesterase activity and suppressing cAMP degradation with a resultant increase in cAMP in platelets and blood vessels, leading to inhibition of platelet aggregation and vasodilation. Cilostazol reversibly inhibits platelet aggregation induced by a variety of stimuli, including thrombin, ADP, collagen, arachidonic acid, epinephrine, and shear stress. The drug is routinely used at doses of 100-200 mg/day.

Oral Hypoglycemic Compounds

One or more oral hypoglycemic compounds, including a biguanide, thiazolidinedione, alpha-glucosidase inhibitor, insulin secretagogue, dipeptidyl peptidase IV inhibitor, or protein kinase C inhibitor can be used in combination with a pFox inhibitor and/or an HMG CoA reductase inhibitor for the treatment of endothelial dysfunction and diabetes.

Biguanides

The biguanides that can be used include metformin and phenformin. These compounds have been well described in the art, e.g. in U.S. Pat. No. 6,693,094. Metformin (N,N-dimethylimidodicarbonimidicdiamide; 1,1-dimethylbiguanide; N,N-dimethylbiguanide; N,N-dimethyldiguanide; N,N-dimethylguanylguanidine) is an anti-diabetic agent that acts by reducing glucose production by the liver and by decreasing intestinal absorption of glucose. It is also believed to improve the insulin sensitivity of tissues elsewhere in the body (increases peripheral glucose uptake and utilization). Metformin improves glucose tolerance in impaired glucose tolerant (IGT) subjects and Type 2 diabetic subjects, lowering both pre- and post-prandial plasma glucose. Metformin is generally not effective in the absence of insulin. Bailey, Diabetes Care 15:755-72 (1992). Metformin (Glucophage™) is commonly administered as metformin HCl. Metformin is also available in an extended release formulation (Glucophage XR™) Dose ranges of metformin are between 10 to 2550 mg per day, and preferably 250 to 2000 mg per day.

Insulin Sensitizers

Thiazolidinediones

Thiazolidinediones that can be used include troglitazone (Rezulin™), rosiglitazone (sold as Avandia™ by GlazoSmithKline), pioglitazone (sold as Actos™ by Takeda Pharmaceuticals North America, Inc. and Eli Lilly and Company), ciglitazone, englitazone, R483 (produced by Roche, Inc.) and pioglitazone.

Such compounds are well-known, e.g., as described in U.S. Pat. Nos. 5,223,522, 5,132,317, 5,120,754, 5,061,717, 4,897,405, 4,873,255, 4,687,777, 4,572,912, 4,287,200, and 5,002,953; and Current Pharmaceutical Design 2:85-101 (1996). The thiazolidinediones work by enhancing insulin sensitivity in both muscle and adipose tissue and to a lesser extent by inhibiting hepatic glucose production. Thiazolidinediones mediate this action by binding and activating peroxisome proliferator-activated receptor-gamma (PPARgamma). Effective doses include troglitazone (10-800 mg/day), rosiglitazone (1-20 mg/day), and pioglitazone (15-45 mg/day). Phase II studies with the glitazone; R483, have been completed and show a significant dose-dependent reduction of HbA1c. R483 has been tested at doses of 5-40 mg/day.

Alpha-Glucosidase Inhibitors

Alpha-glucosidase inhibitors competitively inhibit alpha-glucosidase, which metabolizes carbohydrates, thereby delaying carbohydrate absorption and attenuating post-prandial hyperglycemia. (Clissod, et al., Drugs, 35:214-23 (1988)). This decrease in glucose allows the production of insulin to be more regular, and as a result, serum concentrations of insulin are decreased as are HbA1c levels.

A variety of glucosidase inhibitors are known to one of ordinary skill in the art and described in U.S. Pat. Nos. 6,821,977 and 6,699,904. Preferred glucosidase inhibitors include acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are described in U.S. Pat. Nos. 4,062,950 and 4,174,439 respectively. The glucosidase inhibitor, adiposine, is described in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethy-1)-D-epi-inositol, and the various N-substituted pseudo-aminosugars related thereto, are described in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are described in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]lethoxy]-benzoate, the various derivatives related thereto and pharmaceutically acceptable acid addition salts thereof, are described in U.S. Pat. No. 5,192,772. The glucosidase inhibitor, MDL-25637, 2,6-dideoxy-7-O-.beta.-D-glucopyrano-syl-2,6-imino-D-glycero-L-gluco-hept-itol, the various homodisaccharides related thereto and the pharmaceutically acceptable acid addition salts thereof, are described in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-alpha-D-glucopyranoside sesquihydrate, the deoxy-nojirimycin derivatives related thereto, the various pharmaceutically acceptable salts thereof and synthetic methods for the preparation thereof, are described in U.S. Pat. Nos. 5,157,116 and 5,504,078. The glucosidase inhibitor, salbostatin and the various pseudosaccharides related thereto, are described in U.S. Pat. No. 5,091,524. The daily dose of alpha-glucosidase inhibitors is usually 0.1 to 400 mg, and preferably 0.6 to 300 mg. Effective dosages of both acarbose and miglitol are in the range of about 25 up to about 300 mg/day.

Insulin Secretagogues

Sulfonylureas

Sulfonylureas are a class of compounds that are well-known in the art, e.g., as described in U.S. Pat. Nos. 3,454,635, 3,669,966, 2,968,158, 3,501,495, 3,708,486, 3,668,215, 3,654,357, and 3,097,242. These compounds generally operate by lowering plasma glucose by increasing the release of insulin from the pancreas. Their action is initiated by binding to and closing a specific sulfonylurea receptor (an ATP-sensitive K⁺ channel) on pancreatic beta-cells. This closure decreases K⁺ influx, leading to depolarization of the membrane and activation of a voltage-dependent Ca²⁺ channel. The resulting increased Ca²⁺ flux into the beta-cell, activates a cytoskeletal system that causes translocation of insulin to the cell surface and its extrusion by exocytosis.

Examples of sulfonylureas (with typical daily dosages indicated in parentheses) include acetohexamide (in the range of about 250 up to about 1500 mg), chlorpropamide (in the range of about 100 up to about 500 mg), tolazimide (in the range of about 100 up to about 1000 mg), tolbutamide (in the range of about 500 up to about 3000 mg), gliclazide (in the range of about 80 up to about 320 mg), glipizide (Glucotrol™) (in the range of about 5 up to about 40 mg), glipizide gastrointestinal therapeutic system (GITS) (extended release) (Glucotrol™) (in the range of about 5 up to about 20 mg), glyburide (in the range of about 1 up to about 20 mg), micronized glyburide (in the range of about 0.75 up to about 12 mg), glimepiride (in the range of about 0.5 up to about 8 mg), and AG-EE 623 ZW. In a preferred embodiment, the sulfonylurea is glimepiride in a daily dose range of 0.5 to 4 mg.

Non-Sulfonylureas

Suitable non-sulfonylureas are described in U.S. Pat. Nos. 6,652,838, 6,734,175, and 6,830,759, and include D-phenylalanine derivatives, such as nateglinide (N-[[4-(1-methylethyl)cyclohexyl]carbonyl]-D-phenylalanine) and meglitinides, such as repaglinide. Nateglinide is a fast-acting antidiabetic agent which functions to stimulate insulin production. Meglitinides, are non-sulfonylurea hypoglycemic agents that have insulin secretory capacity. For example, repaglinide appears to bind to ATP-sensitive potassium channels on pancreatic beta cells and thereby increases insulin secretion. For repaglinide, the effective daily dosage may be in the range of about 0.5 mg up to about 16 mg.

Dipeptidyl Peptidase IV Inhibitors

Dipeptidyl peptidase-IV (DPP-IV) inhibitors are potential drugs for the treatment of type 2 diabetes. The original concept that inhibition of DPP-IV would improve glucose tolerance was based on the observation that glucagon-like peptide-1 (GLP-1) is rapidly cleaved and inactivated by the protease DPP-IV (Hoist, J. J. & Deacon, C. F., Diabetes 47:1663-1670 (1998)). Inhibition of this proteolytic inactivation should prolong the action of GLP-1, which is released postprandially from the L-cells in the gut and increases insulin secretion (the ‘incretin’ concept), resulting in improved glucose tolerance. GLP-1 has also been shown to reduce postprandial and fasting glycemia in subjects with type 1 and type 2 diabetes (Ahren, B., BioEssays 20:642-651 (1998))

The potential of using this approach in the treatment of diabetes is illustrated in studies showing that DPP IV-deficient mice (Marguet, et al., Proc. Nat'l Acad. Sci. USA 97:6874-6879 (2000)) and rats (Nagakura, T., et al., Biochem. Biophys. Res. Commun., 284:501-506 (2001)) exhibit increased insulin secretion and glucose tolerance. Furthermore, in diabetic animal models, improved glucose tolerance and insulin response to oral glucose have been demonstrated by several different DPP IV inhibitors (Pederson, et al., Diabetes, 47:1253-12581(1998)).

In another embodiment, DPP-IV inhibitors are used in combination with an HMG CoA reductase inhibitor and/or a pFox inhibitor for the treatment of patients with diabetes or metabolic syndrome and endothelial dysfunction. Suitable DPP IV inhibitors include those compounds described in U.S. Pat. Nos. 6,683,080, 6,861,440, 6,500,804, and U.S. Patent Publication No. 20040224875, including L-threo-isoleucyl pyrrolidide, L-allo-isoleucyl thiazolidide, L-allo-isoleucyl pyrrolidide; and salts thereof or valine pyrrolidide, NVP-DPP728A (1-[[[2-[{5-cyanopyridin-2-yl}-amino]ethyl]amino]-acetyl]-2-cyano-(S)-pyrrolidine) LAF-237 (1-[(3-hydroxy-adamant-1-ylamino)-acetyl]-pyrrolidine-2(S)-carbonitrile); TSL-225 (tryptophyl-1,2,3,4-tetra-hydroisoquinoline-3-carboxylic acid), FE-999011 ([(2S)-1-([2′S]-2′-amino-3-′,3′dimethyl-butanoyl)-pyrrolidine-2-carbonitrile]), GW-229A, 815541, MK-431 or PT-100 (Point Therapeutics). The DP-14 inhibitors may be given at a dosage of from about 0.1-300 mg/kg per day (preferred 1-50 mg/kg per day). Preferred daily doses for NVP DPP728 are 100-300 mg/day.

Combination of Oral Hypoglycemics

In another embodiment, more than one oral hypoglycemic compound is used in combination with a pFox inhibitor and HMG CoA reductase inhibitor. Several of the available oral hypoglycemic agents have been studied in combination and have been shown to further improve glycemic control when compared to monotherapy (Riddle, M., Am. J. Med., 108(suppl 6a): 15S-22S (2000)). As with monotherapy, the choice of a second agent should be based on individual characteristics. Reasonable combinations of agents include a sulfonylurea plus metformin, a sulfonylurea plus an alpha-glucosidase inhibitor, a sulfonylurea plus a thiazolidinedione, metformin plus repaglinide, biguanide plus alpha-glucosidase inhibitor, metformin plus a thiazolidinedione, thiazolidinedione plus DP IV inhibitor, and metformin plus DP IV inhibitor. For example, an oral medication containing metformin plus rosiglitazone is sold as Avandamet™ by GlaxoSmithKline, Inc (in a preferred dose range of from 1 mg/day rosiglitazone/250 mg/day metformin to 8 mg/day rosiglitazone/2,000 mg/day metformin. Oral medications combining glyburide and metformin (Glucovance™) (in a preferred dose range of from 1.25 mg/day glyburide/250 mg/day metformin to 10 mg/day glyburide/2,000 mg/day metformin) and glipizide and metformin (Metaglip™) (in a preferred dose range of from 2.5 mg/day glipazide/250 mg/day metformin to 10 mg/day glipazide/2,000 mg/day metformin) are sold by Bristol Myers Squibb.

In some cases, three oral hypoglycemic compounds, such as sulfonylurea, metformin, thiazolidinedione or sulfonylurea, metformin, alpha-glucosidase inhibitor, may be combined.

Protein Kinase C (PKC) Inhibitors

Recent studies have indicated that the activation of protein kinase C (PKC) and increased diacylglycerol (DAG) levels initiated by hyperglycemia are associated with many vascular abnormalities in retinal, renal, and cardiovascular tissues (Koya, D. & King, G., Diabetes 47:859-866 (1998)). Among the various PKC isoforms, the beta- and delta-isoforms appear to be activated preferentially in the vasculatures of diabetic animals (Inoguchi, et al., Proc. Nat'l Acad. Sci. USA 89:11059-11063 (1992); Ishii, et al., Science 272: 728-731 (1996)), although other PKC isoforms are also increased in the renal glomeruli and retina. The glucose-induced activation of PKC has been shown to increase the production of extracellular matrix and cytokines; to enhance contractility, permeability, and vascular cell proliferation; to induce the activation of cytosolic phospholipase A2; and to inhibit Na+-K+-ATPase. The synthesis and characterization of a specific inhibitor for PKC-beta isoforms has confirmed the role of PKC activation in mediating hyperglycemic effects on vascular cells, and provided in vivo evidence that PKC activation could be responsible for abnormal retinal and renal hemodynamics in diabetic animals (Ishii, et al., Science 272: 728-731 (1996)). Transgenic mice overexpressing PKC-beta isoform in the myocardium developed cardiac hypertrophy and failure, further supporting the hypothesis that PKC-beta isoform activation can cause vascular dysfunctions (Bowman, et al., J. Clin. Invest., 100(9): 2189-2195 (1997)).

In another embodiment, inhibitors of PKC are used in combination with an HMG CoA reductase inhibitor and/or a pFox inhibitor for the treatment of patients with diabetes or metabolic syndrome and endothelial dysfunction. PKC inhibitors, and methods for their preparation are readily available in the art. For example, different kinds of PKC inhibitors and their preparation are described in U.S. Pat. Nos. 5,621,101; 5,621,098; 5,616,577; 5,578,590; 5,545,636; 5,491,242; 5,488,167; 5,481,003; 5,461,146; 5,270,310; 5,216,014; 5,204,370; 5,141,957; 4,990,519; and 4,937,232. Examples of PKC inhibitors include AG 490, PD98059, PKC-alpha/beta pseudosubstrate peptide, staurosporine Ro-31-7549, Ro-31-8220, Ro-31-8425, Ro-32-0432, H-7, sangivamycin; calphostin C, safingol, D-erythro-sphingosine, chelerythrine chloride, melittin; dequalinium chloride, Go6976, Go6983; Go7874, polymyxin B sulfate; cardiotoxin, ellagic acid, HBDDE, 1-O-Hexadecyl-2-O-methyl-rac-glycerol, hypercin, K-252, NGIC-J, phloretin, piceatannol, tamoxifen citrate, flavopiridol, and bryostatin 1. In a preferred embodiment, the inhibitor selectively inhibits the beta- and/or delta-isoforms of PKC. Suitable small molecule PKC-beta inhibitors include LY333531 (developed by Eli Lilly as Ruboxistaurin™). Recent data with this compound from a study of patients receiving 32 mg/day, suggests that ruboxistaurin may have the potential to decrease the progression of diabetic macular edema to involve the center of the macula.

Acetyl-CoA Carboxylase Inhibitors

Acetyl-CoA carboxylase (ACC) catalyzes the rate-limiting reaction in fatty acid biosynthesis (Kim, K. H., Annu. Rev. Nutr., 17, 77-99 (1997); Munday, M. R., & Hemingway, C. J., Adv. Enzyme Reg., 39, 205-234 (1999)). In animals, including humans, there are two isoforms of acetyl-CoA carboxylase expressed in most cells, ACCI (Mr about. 265,000) and ACC2 (Mr about 280,000), which are encoded by two separate genes and display distinct tissue distribution. Both ACC1 and ACC2 produce malonyl-CoA, which inhibits mitochondrial fatty acid oxidation through feedback inhibition of carnitine palmitoyltransferase 1 (CPT-1) (McGarry, J. D., et al., Diabetes Metabol. Revs. 5, 271-284 (1989); and McGarry, J. D., & Brown, N. F., Eur. J. Biochem. 244, 1-14 (1997)), and therefore plays key roles both in controlling the switch between carbohydrate and fatty acid utilization in liver and skeletal muscle and also in regulating insulin sensitivity in the liver, skeletal muscle, and adipose tissue (McGarry, J. D., et al., Diabetes Metabol. Revs. 5, 271-284 (1989); McGarry, J. D., & Brown, N. F., Eur. J. Biochem. 244, 1-14 (1997)). Malonyl-CoA may also play an important regulatory role in controlling insulin secretion from the pancreas (Chen, S., et al., Diabetes, 43, 878-883 (1994)).

Thus, in addition to inhibition of fatty acid synthesis, reduction in malonyl-CoA levels through ACC inhibition may provide a mechanism for increasing fatty acid utilization that may reduce TG-rich lipoprotein secretion (very low density lipoprotein) by the liver, alter insulin secretion by the pancreas, and improve insulin sensitivity in liver, skeletal muscle, and adipose tissue. Additionally, by increasing fatty acid utilization and by preventing increases in de novo fatty acid synthesis, chronic administration of an ACC inhibitor may also deplete liver and adipose tissue TG stores in obese subjects consuming a low fat diet, leading to selective loss of body fat.

Therefore, an ACC inhibitor can be used to effectively and simultaneously treat the multiple risk factors associated with metabolic syndrome and could have a significant impact on the prevention and treatment of the cardiovascular morbidity and mortality associated with obesity, hypertension, diabetes, and atherosclerosis. In another embodiment, ACC inhibitors are used in combination with an HMG CoA reductase inhibitor and/or a pFox inhibitor for the treatment of patients with diabetes or metabolic syndrome and endothelial dysfunction. Examples of suitable acetyl-CoA carboxylase inhibitors are described in U.S. Pat. Nos. 6,734,337 and 6,485,941 and in Harwood et al. J. Biol. Chem., Vol. 278, Issue 39, 37099-37111 (2003). These include compounds such as the isozyme-nonselective ACC inhibitors CP-640186 and CP-610431.

Rho-Kinase Inhibitors

Increased activity of Rho-kinase causes hypercontraction of vascular smooth muscle and has been implicated as playing a pathogenetic role in divergent cardiovascular diseases such as coronary artery spasm. Vasospastic angina is a form of angina caused by coronary artery spasm. Compounds which inhibit rho-kinase can be used to treat this form of angina. Suitable compounds include the selective rho-kinase inhibitor fasudil.

The combination of an HMG CoA reductase inhibitor, such as a statin (e.g., “simvastatin”), in combination with a pFox inhibitor, such as trimetazidine (“Simetazidine”), is beneficial for treatment of acute coronary syndrome (ACS) and chronic angina, particularly in diabetics. HDL activates eNOS and both simvastatin and atorvastatin increase HDL, with atorvastatin more than simvastatin. Trimetazidine also raises HDL, and may be therapeutic by virtue of being an agonist of eNOS, as well as being a pFOX inhibitor. Accordingly, part of the benefit of the treatment of acute coronary syndrome is the lowering of CRP.

This combination is useful for the treatment of these conditions in diabetic and non-diabetic patients. In patients with diabetes, especially Type II diabetes, the addition of one or more oral hypoglycemic compound to the pFox inhibitor and HMG CoA reductase inhibitor is particularly advantageous to control glucose levels. The combinations can also be used to treat patients who cannot take beta blockers, such as those suffering from sick sinus syndrome (slow heart rhythms) and other conduction system disturbances as well as those patients suffering from asthma and chronic obstructive lung diseases accompanied by bronchospasm.

The combination therapy is also useful in the treatment and/or prevention of metabolic syndrome, a collection of major and emerging cardiovascular risk factors that stem from underlying insulin resistance. Metabolic syndrome is a common precursor to both atherosclerotic vascular disease (ASCVD) and type II diabetes. Metabolic syndrome likely develops from obesity, physical inactivity, and atherogenic diet, although a genetic predisposition may contribute. These factors lead to insulin resistance, which, in turn, contribute to a typical set of major and emerging risk factors: abdominal obesity; elevated blood pressure; atherogenic dyslipidemia (high triglycerides, low HDL, and small, dense LDL); impaired fasting glucose or glucose intolerance; proinflammatory state; and prothrombotic state. By definition three or more of these risk factors constitutes the metabolic syndrome.

The statin is preferably given in a dose of between 5 and 80 mg/day in two or three separate doses. In a preferred embodiment, the pFox inhibitor is administered in a dosage of between 5 and 1000 mg/day, more preferably between 10 and 100 mg/day, most preferably between 60 and 90 mg/day. In a more preferred embodiment, the pFox inhibitor trimetazidine is administered in a sustained or extended dosage formulation at a dose of 45 mg two times a day or in an immediate release formulation at a dose of 20 mg three times a day.

Examples of suitable combinations include 13.33 mg simvastatin with 20 mg of trimetazidine given three times a day; 20 mg simvastatin with 45 mg of extended release trimetazidine given twice daily; 26.66 mg atorvastatin with 20 mg of trimetazidine given three times a day; 40 mg atorvastain with 45 mg of extended release trimetazidine given twice a day; 10 mg of simvastatin with 250 mg of mildronate given twice daily (two tablets); 20 mg simvastatin with 250 mg mildronate one daily (one tablet); and 20 mg atorvastatin with 250 mg mildronate given twice daily (1-2 tablets). Statin-mildronate combinations can also be administered intravenously, which in combination with a statin, for example, pravastatin i.v., may be useful for treatment of acute coronary syndrome.

If the statin is simvastatin, the most preferred administration regime is 20 mg of simvastatin combined in a single tablet or capsule with 45 mg of trimetazidine extended release and dosed twice daily. If the statin is atorvastatin, the most preferred regime is 40 mg of atorvastatin combined in a single tablet or capsule with 45 mg of trimetazidine extended release and dosed twice daily.

If an oral hypoglycemic is added to the combination of pFox inhibitor and HMG CoA reductase inhibitor, preferred drugs and doses include glimepiride, administered in a dose of from 0.5 to 4 mg/day; glipizide, administered in a dose of from 5 to 20 mg/day; rosaglitazone, administered in a dose of from 100 mg to 600 mg/day; metformin, administered in a dose of from 250 to 2000 mg/day; a combination of glipizide and metformin administered in a dose from 2.5 mg/day glipazide/250 mg/day metformin to 10 mg/day glipazide/2,000 mg/day metformin; a combination of glyburide and metformin administered in a dose of from 1.25 mg/day glyburide/250 mg/day metformin to 10 mg/day glyburide/2,000 mg/day metformin; and a combination of rosaglitazone and metformin administered in a dose of from 1 mg/day rosiglitazone/250 mg/day metformin to 8 mg/day rosiglitazone/2,000 mg/day metformin. In another embodiment, a combination of a nitric oxide generator and a pFox inhibitor which results in a non-hemodynamic interaction is administered to improve oxygen utilization by the myocardium.

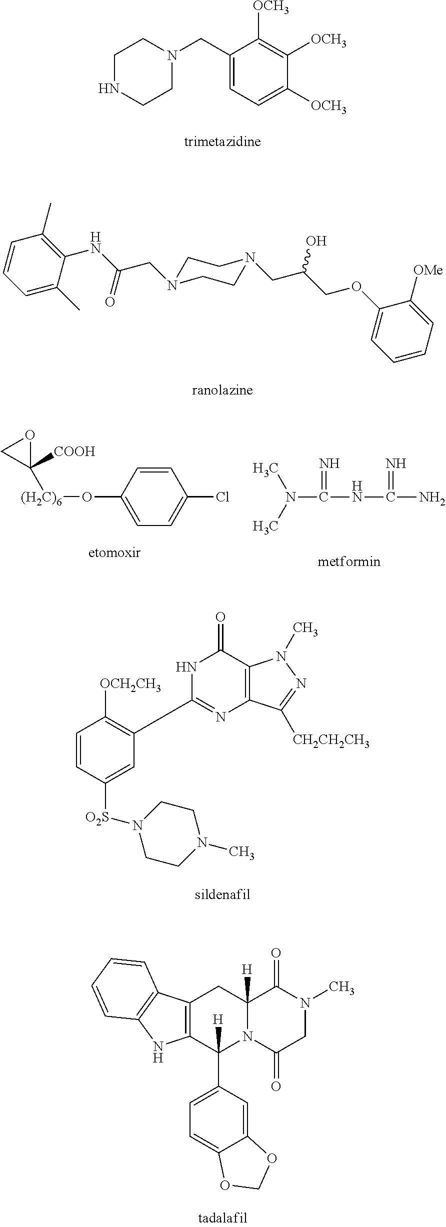

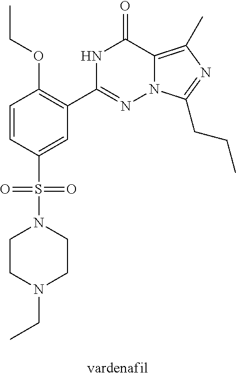

The teachings of all patents, published applications and references cited herein are incorporated by reference in their entirety. While this invention has been particularly shown and described with references to example embodiments thereof, it will be understood by those skilled in the art that various changes in form and details may be made therein without departing from the scope of the invention encompassed by the appended claims.

1-13. (canceled)

14. A method for treating type 2 diabetes mellitus comprising administering to a subject in need thereof a therapeutically effective amount of an inhibitor of fatty acid oxidation.

15. A method for treating hyperglycemia comprising administering to a subject in need thereof a therapeutically effective amount of an inhibitor of fatty acid oxidation.

16. A method for treating impaired glucose tolerance comprising administering to a subject in need thereof a therapeutically effective amount of an inhibitor of fatty acid oxidation.

17. The method of claim 14, wherein the inhibitor of fatty acid oxidation is trimetazidine.

18. The method of claim 17, wherein the method is a method of front line therapy.

19. A method for treating type 2 diabetes mellitus comprising administering to a subject in need thereof a therapeutically effective amount of an inhibitor of fatty acid oxidation and an inhibitor of hepatic glucose output.

20. A method for treating hyperglycemia comprising administering to a subject in need thereof a therapeutically effective amount of an inhibitor of fatty acid oxidation and an inhibitor of hepatic glucose output.

21. A method for treating impaired glucose tolerance comprising administering to a subject in need thereof a therapeutically effective amount of an inhibitor of fatty acid oxidation and an inhibitor of hepatic glucose output.

22. The method of claim 19, wherein the inhibitor of fatty acid oxidation is a 3-KAT inhibitor.

23. The method of claim 22, wherein the 3-KAT inhibitor is trimetazidine.

24. The method of claim 19, wherein the inhibitor of hepatic glucose output is metformin.

25. The method of claim 19, wherein the inhibitor of fatty acid oxidation is trimetazidine and the inhibitor of hepatic glucose output is metformin.

26. The method of claim 25, wherein trimetazidine is administered at about 30 mg/day to about 180 mg/day, and metformin is administered at about 1000 mg/day to about 2550 mg/day.

27. A method for treating diabetic retinopathy comprising administering to a subject in need thereof a therapeutically effective amount of an inhibitor of fatty acid oxidation.

28. The method of claim 27, wherein said diabetic retinopathy is in association with type 2 diabetes mellitus or hyperglycemia.

29. The method of claim 27, wherein said diabetic retinopathy is not associated with diabetes mellitus.

30. The method of claim 27, wherein the inhibitor of fatty acid oxidation is a 3-KAT inhibitor. 3 L (Original) The method of claim 30, wherein the 3-KAT inhibitor is trimetazidine.

32-34. (canceled)

35. A method for treating diabetic nephropathy comprising administering to a subject in need thereof a therapeutically effective amount of an inhibitor of fatty acid oxidation.

36. The method of claim 35, wherein said diabetic nephropathy is characterized by impaired glomerular filtration, microalbuminuria, proteinuria or any combination of the foregoing.

37. The method of claim 35, wherein said diabetic nephropathy is associated with type 2 diabetes mellitus or hyperglycemia.

38. The method of claim 35, wherein said diabetic nephropathy is not associated with diabetes mellitus.

39. The method of claim 35, wherein the inhibitor of fatty acid oxidation is a 3-KAT inhibitor.

40. The method of claim 39, wherein the 3-KAT inhibitor is trimetazidine.

41-43. (canceled)

44. A method for treating diabetic neuropathy comprising administering to a subject in need thereof a therapeutically effective amount of an inhibitor of fatty acid oxidation.

45-51. (canceled)

52. A pharmaceutical composition comprising trimetazidine and metformin and a physiologically acceptable carrier.

53. A kit for the treatment of hyperglycemia, type 2 diabetes mellitus, impaired glucose tolerance, diabetic retinopathy, diabetic nephropathy and/or diabetic neuropathy comprising a first pharmaceutical composition comprising an inhibitor of fatty acid oxidation; a second pharmaceutical composition comprising an inhibitor of hepatic glucose output or an antidiabetic agent; and a container for containing said first pharmaceutical composition and said second pharmaceutical composition.

54-65. (canceled)
